(19) United States
(12) Patent Application Publication
(10) Pub. No.: US 2022/0202050 A1
(43) Pub. Date: Jun. 30, 2022

(54) SOLID FOOD COMPOSITION

(71) Applicant: Gloobe PTE LTD, Singapore (SG)

(72) Inventors: Gunnar Norstedt, Bromma (SE); Micael Györei, Limhamn (SE); Magnus Sederholm, Gränna (SE); Thomas Buchar, Jönköping (SE)

(73) Assignee: Gloobe PTE LTD, Singapore (SG)

(21) Appl. No.: 17/607,130

(22) PCT Filed: Apr. 27, 2020
(86) PCT No.: PCT/EP2020/061607

§ 371 Date: Oct. 28, 2021

(30) Foreign Application Priority Data
Apr. 29, 2019 (EP) 19171558.0

(51) Int. Cl. A23L 7/10; A23L 7/20; A23L 5/20; A23L 33/10; A23L 33/00; A61K 36/899; A61P 3/10

(52) U.S. Cl. CPC A23L 7/197; A23L 7/20; A23L 5/20; A23L 33/10; A61K 2236/37; A61K 36/899; A61P 3/10; A23V 2002/00; A23L 33/40

(57) ABSTRACT

The present invention relates to a solid food composition which is generally gluten and lactose free, and its use for treating and preventing metabolic diseases.

### TECHNICAL FIELD

The present invention relates to a solid food composition which is generally gluten and lactose free and is used for treating and preventing metabolic diseases.

### BACKGROUND

Macro-nutrient preload means the ingestion of a small nutritional load approximately one-half hour before regular meals. The preload activates the gastro-intestinal (GI) system and this includes the release of incretins such as glucagon-like peptide-1 (GLP-1). The preload activated GI signals will in turn activate insulin, the main hormone required for glucose uptake in cells. The net effect of preload in a human subject is therefore to reduce the increased blood glucose following a meal. This situation is of relevance for clinical conditions characterized by increased glucose levels e.g. different types of diabetes but also other conditions featuring particular metabolic alterations e.g. polycystic ovary syndrome (PCOS). The significance of GLP-1 in diabetes is further substantiated by the situation that pharmacological compounds that increase GLP-1 are clinically used for the treatment of diabetes. In contrast to pharmacological compounds, preload treatment is a natural way to increase GLP-1.

A preload response can be evoked by all major macro-nutrients i.e. fat, carbohydrates and proteins and this response, besides the incretin response, also has a neuronal component where nerve signals are activated by chewing leading to a metabolic awareness that food is to be delivered to the GI system (Miquel-Kergoat et al., 2015).

In summary, the preload response is activated by a variety of nutrients and the knowledge of this response has led to the suggestion that preload is a safe and simple treatment paradigm for diabetes and diabetes associated conditions. There is a need to develop safe and simple diabetes treatment since diabetes is taking world epidemic proportions. It is envisioned that preload treatment has the potential to be a first line treatment for pre-diabetic states and to, at least partly, substitute for pharmacological treatments (GLP-1 analogues, oral anti-diabetic compounds and insulin), which should be limited especially in patients affects by gestational diabetes mellitus (GBM). It is therefore important to optimize the preload composition as well as to implement new and innovative ways to manufacture preload for the above-mentioned indications.

### SUMMARY

The present invention concerns a solid food composition, also referred to herein as a preload product, which has beneficial effects on the state of gestational diabetes mellitus (GDM) and obesity/overweight. The preload product is composed of natural, non-animal ingredients and may e.g. be in the form of a biscuit or cracker. When the solid food composition is in the form of a biscuit, it may also be referred to as a preload biscuit. The mode of action of this product is a rapid effect on blood glucose levels evoked by the activation of incretins. In addition, treatment with preload leads to long-term effects thanks to its ability to reduce inflammation, alter the gastro-intestinal microflora and enhance the gut barrier function. By using the preload product, women diagnosed with GDM and/or obesity will benefit from improved glycaemic state and reduced inflammation and body weight. Thereby their metabolic situation will improve leading to a healthier pregnancy and a reduction of the complications due to GDM and obesity.

One aspect of the present disclosure relates to a solid food composition comprising: A solid food composition comprising:
A protein in a range between 3 and 14% dry weight,
A lipid in a range between 8 and 22% dry weight,
A complex carbohydrate in a range between 35 and 80% dry weight,
soluble fibers in a range between 2 and 19% dry weight
   wherein the complex carbohydrate has been treated to comprise a reduced amount of phytic acid compared to the untreated carbohydrate, and wherein all ingredients of the composition are of plant origin.

The invention also provides a food composition that can be used as a drug vehicle.

The invention further provides a method for manufacturing a solid food composition, the method comprising:
A. Providing cereal grains;
B. Treating said grains to reduce their phytic acid content;
C. Subjecting said grains to a step of heat treatment;
D. Finely dividing said grains
E. Adding at least one liquid and one or more additional ingredients to said finely divided grains to obtain a slurry
F. Incubating the slurry at a low temperature in the range of between 60° C. and 95° C. for a time interval in the range of between 30 and 180 minutes; and
G. Incubating the slurry at a high temperature in the range of between 125° C. to 140° C. for a time interval in the range of between 1 and 10 minutes;
wherein step B. may be performed at any time during the method and steps F. and G. may be performed in any order, thereby obtaining a solid food composition.

One aspect of the present disclosure relates to the use of a solid food composition as disclosed herein for increasing satiety, increasing the sense of fullness, and/or reducing appetite.

One aspect of the present disclosure relates to a solid food composition disclosed herein for use as a medicament.

One aspect of the present disclosure relates to a solid food composition disclosed herein for use in the treatment or prevention of a metabolic disorder.

One aspect of the present disclosure relates to a method for treating or preventing a metabolic disorder in an individual comprising administering to the individual an effective amount of the solid food composition disclosed herein.

One aspect of the present disclosure relates to a method for manufacturing a solid food composition, the method comprising:
a) Providing a protein in a range between 3 and 14% dry weight, a lipid in a range between 8 and 22% dry weight, and a complex carbohydrate in a range between 35 and 75% dry weight;
b) Treating the complex carbohydrate to reduce its phytic acid content;
c) Mixing the ingredients of a) and b) to form a slurry;
d) Heating the slurry first to a temperature in the range of between 125° C. to 140° C. for a time interval in the range of between 1 and 10 minutes; and
e) Reducing then the temperature to between 70° C. and 95° C., and keeping it for a time interval in the range of between 50 and 180 minutes,

thereby obtaining a solid food composition.

One aspect of the present disclosure relates to a container comprising at least one solid food composition as defined in any one of the preceding claims, wherein the at least one solid food composition is in a package.

### DESCRIPTION OF DRAWINGS

FIG. 1. A schematic drawing of a mobile phone application that may be used together with the preload treatment.

FIG. 2. A: effect of preload (18.9 g carbohydrates) on a standard oral glucose tolerance test (OGTT); B: effect of preload (25 g carbohydrates) on a OGTT.

FIG. 3. Outline of a gestational diabetes mellitus (GDM) feasibility test.

FIG. 4. Outline of a GDM clinical trial (regular clinical trial GDM).

FIG. 5. Time-temperature matrix for baking and stabilization of a preload biscuit.

FIG. 6 shows the lipase activity in oats, germinated oats, dried oats and microwaved oats.

FIG. 7 shows the lectin content as determined by a lectin-hemagglutination test.

FIG. 8 shows the blood glucose level (mmol) in 3 volunteers after 8 hours fasting followed by intake of 2 Preload biscuits (Test 1), intake of water (Control 1), intake of 2 Preload biscuits followed by intake of 25 g glucose after 30 min. (Test 2) or intake of 25 g glucose (Control 2).

### DETAILED DESCRIPTION

### Definitions

The term “low glycemic index (GI)” as used herein refers to a value assigned to foods based on how slowly or how quickly those foods cause increases in blood glucose levels. GI and glycemic load (GL) are measures of the effect on blood glucose level after a food containing carbohydrates is consumed. Glucose has a glycemic index of 100 units, and all foods are indexed against that number. A low GI is a GI of 55 or less; a medium GI is a GI between 56 and 69; a high GI is a GI of 70 or more. Low GI foods affect blood glucose and insulin levels less and have a slower rate of digestion and absorption. The glycemic load (GL) of food is a number that estimates how much the food will raise a person's blood glucose level after eating it. One unit of glycemic load approximates the effect of consuming one gram of glucose. Glycemic load accounts for how much carbohydrate is in the food and how much each gram of carbohydrate in the food raises blood glucose levels. Glycemic load is based on the glycemic index (GI), and is calculated by multiplying the grams of available carbohydrate in the food times the food's GI and then dividing by 100. Throughout the present application, the glycemic load indicated as grams/day.

The term “complex carbohydrate” as used herein refers to a carbohydrate molecule comprising at least three monosaccharide molecules bound to form a chain. A complex carbohydrate is usually a long chain of monosaccharides, such as starch and cellulose. Opposite to complex carbohydrates are simple carbohydrates, which are mono and disaccharides.

The term “fiber” as used herein refers to dietary fiber, which is the indigestible portion of food derived from plants. Dietary fiber can be soluble or insoluble. Soluble fiber dissolves in water, is readily fermented in the colon into gases and physiologically active by-products, and can be prebiotic and viscous. Insoluble fiber does not dissolve in water, is metabolically inert and provides bulking, or it can be fermented in the large intestine. Fibers are a group of compounds defined as being non-starch polysaccharides, for example arabinoxylans, cellulose, resistant starch, resistant dextrins, inulin, lignin, chitins, pectins, beta-glucans, oligosaccharides and other plant components.

The term “incretins” as used herein refers to a group of metabolic hormones that stimulate a decrease in blood glucose levels. The two main candidate molecules that fulfil criteria for an incretin are the intestinal peptides glucagon-like peptide-1 (GLP-1) and gastric inhibitory peptide (also known as: glucose-dependent insulinotropic polypeptide or GIP). Incretins cause secretion of insulin from pancreatic beta cells of the islets of Langerhans by a blood glucose-dependent mechanism.

The term “neural response” as used herein refers to cephalic phase responses activating neuronal pathways which influence satiation processes and lead to a reduction in overeating. For example, the neural response can activate the incretin hormonal system and thereby result in release of incretins, such as GLP-1.

The term “dysbiotic microbiota” as used herein refers to a microbiota that is not normal. Dysbiotic microbiota is characterized by having low gene and phylae richness. Normal microbiota is characterized by having a large gene and phylae richness. Normal microbiota is characterized by comprising bacteria belonging to the genera Bacterioidetes, Faecalibacterium, Roseburia, Blautia, Ruminococcus, Coprococcus, Bifidobacterium, Methanobrevibacter, Lactobacillus, Coprococcus, Clostridium, Akkermansia, Eubacterium.

The term “pseudocereal” as used herein refers to a plant classified as a non-grass that can be used in the same way as cereals (cereals are grasses). For example, their seed can be ground into flour and used as cereals.

The terms “treatment” and “treating” as used herein refer to the management and care of a patient for the purpose of combating a condition, disease or disorder. The term is intended to include the full spectrum of treatments for a given condition from which the patient is suffering, such as administration of the active compound for the purpose of: alleviating or relieving symptoms or complications; delaying the progression of the condition, disease or disorder; curing or eliminating the condition, disease or disorder; and/or preventing the condition, disease or disorder, wherein “preventing” or “prevention” is to be understood to refer to the management and care of a patient for the purpose of hindering, reducing or delaying the development of the condition, disease or disorder, and includes the administration of the active compounds to prevent or reduce the risk of the onset of symptoms or complications. The patient to be treated is preferably a mammalian, in particular a human being. The patients to be treated can be of various ages.

Solid Food Composition

One aspect of the present disclosure relates to a composition comprising a solid food composition comprising:
a protein in a range between 3 and 18%, for example in a range between 3 and 14% dry weight,
a lipid in a range between 8 and 25%, for example in a range between 8 and 22% dry weight,
a complex carbohydrate in a range between 35 and 80% dry weight,
soluble fibers in a range between 2 and 19% dry weight
   wherein the complex carbohydrate has been treated to comprise a reduced amount of phytic acid compared to the untreated carbohydrate, and
   wherein all ingredients of the composition are of plant origin.

Several of the ingredients of the solid food composition may be comprised within cereal grains, preferably oat grains. Cereal grains, in particular oat grains comprises both proteins and complex carbohydrates. Thus, it is comprised within the invention that said solid food composition comprises cereal grains, such as oat grains. Said cereal grains have preferably been treated to reduce the level of phytic acid.

For example the solid food composition may be prepared from the following ingredients:
cereal grains, preferably oat grains, preferably oat grains treated to reduce the content of phytic acid and/or lectins as described below
water
optionally salt
a sweetener, preferably a sweetener with a low glycemic index
a vegetable oil.

In addition to above-mentioned ingredient other components may be added, for example with the aim of achieving a particular effect. Such components include flavors, vitamins and drugs as described in more detail below.

Phytic acid, also known as inositol hexakisphosphate (IP6), inositol polyphosphate or phytate when in salt form, is the principal storage form of phosphorus in many plant tissues, especially bran and seeds. Phytic acid, mostly as phytic acid in the form of phytin, is found within the hulls of seeds, including nuts, grains and pulses. Phytic acid has a strong binding affinity to important minerals, such as calcium, iron, and zinc. When iron and zinc bind to phytic acid they form insoluble precipitates and are far less absorbable in the intestines. This process can therefore contribute to iron and zinc deficiencies. Thus, it is beneficial to reduce phytic acid content of food.

The complex carbohydrate may be comprised within grains, for example within cereal grains, preferably within oat grains. Thus, the solid compositions preferably comprises cereal grains, e.g. processed cereal grains. Thus, treating said complex carbohydrate to reduce the amount of phytic acid, may comprise or consist of a treatment of the cereal grains, e.g. oat grains used for preparation of the food composition. The inventors have found that the treatment applied to the complex carbohydrate to reduce the phytic acid content also results in a reduction of the lectins content of said carbohydrate.

Thus, in one embodiment of the present disclosure the complex carbohydrate comprises a reduced amount of lectins compared to the untreated carbohydrate.

Lectins are carbohydrate-binding proteins, macromolecules that are highly specific for sugar moieties. Lectins are one of many toxic constituents of many raw plants, which are inactivated by proper processing and preparation. Lectins are toxic for animals, and thus humans, if consumed at high doses.

In one embodiment the present disclosure relates to a solid food composition comprises potato (Solanum tuberosum) protein in a range between 4 to 8% dry weight, such as between 4 and 7% dry weight, such as between 4 and 6% dry weight; coconut oil in a range between 12 to 18% dry weight, such as between 12 and 16% dry weight, such as between 12 and 14% dry weight, such as between 15 and 18% dry weight; and oat in a range between 60 to 70% dry weight, such as in a range between 65 to 70% dry weight, such as in a range between 60 to 65% dry weight. The at least three ingredients are present in the composition in such an amount that their sum is at the most 100%.

In one embodiment of the present disclosure, the composition comprises a low level of phytic acid. Thus, the complex carbohydrate of the composition disclosed herein has been treated to reduce its phytic acid content, as described below in the section “Method of manufacturing a solid food composition”.

In one embodiment it is preferred that the solid food composition has a level of phytic acid below 5‰, preferably below 4‰, such as below 3‰.

As described elsewhere, it is preferred that one ingredient of the solid compositions of the invention is cereal grains, such as oat grains treated to reduce the content of phytic acid. It is preferred that the treated cereal grains, such as oat grains used for manufacture of the solid food compositions of the invention comprises at the most 0.8 g, preferably at the most 0.6 g, such as at the most 0.5 g phytic acid per 100 g dry weight.

In one embodiment of the present disclosure, the composition comprises a low level of lectins. Thus, the complex carbohydrate of the composition disclosed herein has been treated to reduce its lectins content, as described below in the section “Method of manufacturing a solid food composition”.

In one embodiment of the present disclosure, the composition comprises a protein in a range between 2 and 18% dry weight, for example in a range between 3 and 14% dry weight, such as in a range between 5 and 14% dry weight, such as in a range between 6 and 14%, such as in a range between 8 and 14% dry weight, such as in a range between 10 and 14% dry weight, such as in a range between 12 and 14% dry weight, such as in a range between 3 and 12% dry weight, such as in a range between 3 and 10% dry weight, such as in a range between 3 and 8% dry weight, such as in a range between 3 and 8% dry weight, such as in a range between 3 and 5% dry weight.

At least 3% of protein, such as at least 5% of protein is needed for the solid food composition to induce incretins production.

In one embodiment of the present disclosure, the composition comprises a lipid, preferably a vegetable oil in a range between 8 and 25% dry weight, for example in a range between 8 and 22% dry weight, such as in a range between 8 and 20% dry weight, such as in a range between 8 and 17% dry weight, such as in a range between 8 and 15% dry weight, such as in a range between 8 and 12% dry weight, such as in a range between 8 and 10% dry weight, such as in a range between 10 and 22% dry weight, such as in a range between 12 and 22% dry weight, such as in a range between 15 and 22% dry weight, such as in a range between 17 and 22% dry weight, such as in a range between 20 and 22% dry weight.

In one embodiment of the present disclosure, the composition comprises a complex carbohydrate in a range between 35 and 80% dry weight, such as in a range between 35 and 75% dry weight, such as in a range between 35 and 70% dry weight, such as in a range between 35 and 65% dry weight, such as in a range between 35 and 60% dry weight, such as in a range between 35 and 55% dry weight, such as in a range between 35 and 50% dry weight, such as in a range between 35 and 45% dry weight, such as in a range between 35 and 40% dry weight, such as in a range between 40 and 80% dry weight, such as in a range between 50 and 80% dry weight, such as in a range between 55 and 80% dry weight, such as in a range between 60 and 80% dry weight, such as in a range between 65 and 80% dry weight, such as in a range between 70 and 80% dry weight.

The sum of the percentage of the protein, the carbohydrate and the lipid is at most 100%.

In one embodiment of the present disclosure, the complex carbohydrate has been treated to comprise a reduced amount of lectins compared to the untreated carbohydrate.

In one embodiment of the present disclosure, the protein has been treated to comprise a reduced amount of lectins compared to the untreated protein.

Reduction of phytic acid and/or lectins is described in the section below “Method of manufacturing a solid food”.

In one embodiment of the present disclosure, the solid food composition as disclosed herein does not comprise ingredients deriving from animals.

The protein may be protein from cereal grains, in particular from oat grains. Thus, in one embodiment, the protein is not added separately to the solid food composition, but is comprised within cereal grains, e.g. oat grains, which have been treated to reduce the level of phytic acid.

It is however also comprised within the invention that the protein is a protein isolate. In one embodiment of the present disclosure, the solid food composition as disclosed herein comprises a protein ingredient and said protein is protein isolate obtained from a tuber, a seed or a legume.

In one embodiment of the present disclosure, the protein is protein isolate obtained from potatoes (Solanum tuberosum), oat, hemp, peas, beans, lentils, soy, quinoa, amaranth, breadnut, chia, kaniwa, spirulina and nuts.

In one embodiment of the present disclosure, the protein ingredient is protein isolate obtained from potatoes (Solanum tuberosum), oat or hemp.

In one embodiment of the present disclosure, the protein ingredient is protein isolate from potatoes (Solanum tuberosum).

In one embodiment of the present disclosure, the protein ingredient is protein isolate from oat.

In one embodiment of the present disclosure, the solid food composition as disclosed herein comprises a lipid and said lipid may in particular be a vegetable oil.

In one embodiment of the present disclosure, the lipid is a vegetable oil, wherein the vegetable oil preferably is selected from a group consisting of coconut oil, sunflower oil, rapeseed oil, canola oil, peanut oil, corn oil, palm oil, avocado oil, walnut oil, brassica oil, olive oil and linseed oil.

In one embodiment of the present disclosure, the lipid is vegetable oil, wherein the vegetable oil is coconut oil.

The complex carbohydrate may comprise soluble fibers. In one embodiment of the present disclosure, the complex carbohydrate comprises β-glucans (β(1,3)(1,4)-glucans). Thus, in a preferred embodiment of the invention, the solid composition according to the invention comprises beta-glucan. In fact, β-glucans improve blood glucose regulation even in individuals affected by hypercholesterolemia. Examples of cereals rich in β-glucans are oat, barley, wheat, and rye. Said beta-glucan may be comprised within an ingredient of the solid composition, e.g. said beta-glucan may be comprised within cereal grains, such as oat grains. However, beta-glucan may also be added separately to the solid food composition. Thus, in one embodiment of the present disclosure, the solid food composition comprises a β-glucans concentrate. It is also comprised within the invention, that the solid composition may comprise beta-glucans from several sources, e.g. beta-glucans comprised in cereal grains, such as oat grains used for manufacture of the solid composition as well as a beta-glucan concentrate.

In one embodiment of the present disclosure, the solid food composition comprises dry-fractionated high molecular weight β-glucans concentrate. For example, the solid food composition of the present disclosure may comprises dry-fractionated high molecular weight β-glucans concentrate, which has been treated to reduce its phytatic acid content.

In one embodiment of the present disclosure, the solid food has a β-glucans content of at least 5% by weight (w/w), such as of at least 6% by weight (w/w), such as of at least 7% by weight (w/w).

A high β-glucans content results in an increased viscosity in the gut, which delays absorption of carbohydrates and attenuates the blood sugar level response, so that large fluctuations in blood glucose level are minimized and/or prevented. Moreover, the delayed absorption of carbohydrates causes a large portion of the injected food to reach the colon and thereby to exert a positive action of the colon microbiome (probiotic effect) and to lower systemic inflammation.

In one embodiment of the present disclosure, the complex carbohydrate is a cereal or a pseudocereal.

In one embodiment of the present disclosure, the complex carbohydrate is a cereal or a pseudocereal selected from a group consisting of oat, corn, rice, millets and buckwheat, wheat, such as kamut and spelt, barley, quinoa and amaranth.

In one embodiment of the present disclosure, the complex carbohydrate is a gluten free cereal or pseudocereal.

In one embodiment of the present disclosure, the complex carbohydrate is oat.

In one embodiment of the present disclosure, the composition of the present disclosure comprises or consists of oat in a range between 55 to 70% dry weight, coconut oil in a range between 12 to 18% dry weight and β-glucans concentrate in a range between 10 and 20% by weight.

In one embodiment of the present disclosure, the composition of the present disclosure comprises a β-glucans concentrate.

In one embodiment of the present disclosure, the β-glucans concentrate comprises soluble and insoluble fibers, and wherein the soluble fibers are at least 20% by weight of the β-glucans concentrate.

In one embodiment of the present disclosure, the soluble fibers of the β-glucans concentrate comprise at least 20% by weight of high molecular weight β-glucans, such as at least 25% by weight of high molecular weight β-glucans.

In one embodiment of the present disclosure, the high molecular weight β-glucans have a weight average molecular weight of 30.000 g/mol or higher, such as of 50.000 g/mol or higher. For example the high molecular weight β-glucans may have a weight average molecular weight comprised between 35.600 and 650.000 g/mol. For example the high molecular weight β-glucans may have a number average molecular weight comprised between 30.200 and 481.000 g/mol.

In one embodiment of the present disclosure, the β-glucans concentrate fibers comprises at least 50% by weight insoluble fibers.

In one embodiment of the present disclosure, the complex carbohydrate is a gluten free cereal or pseudocereal selected from the group consisting of oat, corn, rice, amaranth, quinoa, millets and buckwheat.

In one embodiment of the present disclosure, the solid food composition comprises fibers in a range between 11 and 19% dry weight, such as between 11 and 17% dry weight, such as between 11 and 15% dry weight, such as between 11 and 13% dry weight, such as between 12 and 14% dry weight, such as between 13 and 15% dry weight, such as between 14 and 17% dry weight, such as between 13 and 18% dry weight, such as between 15 and 19% dry weight and said fibers comprise at least 50% by weight insoluble fibers. These insoluble fibers give prebiotic characteristics to the solid food composition.

In one embodiment of the present disclosure, the solid food composition has a low glycemic index (GI). For example, in one embodiment of the present disclosure the solid food composition has a glycemic index below 55.

In one embodiment of the present disclosure, the solid food composition further comprises water soluble vitamins and/or lipid soluble vitamins. Examples of water soluble vitamins are the vitamins of the B-group and vitamin C. Examples of fat soluble vitamins are vitamin A, D, E and K.

In one embodiment of the present disclosure, the solid food composition further comprises a sweetener. The sweetener is preferably a sweetener with a low glycemic index, such as agave syrup. Preferably, the sweetener has a glycemic index comparable to or lower than agave syrup, such as a glycemic index which is at the most 10% higher than the glycemic index of agave syrup. In one embodiment, the solid food composition may comprise agave syrup.

The solid food composition may further comprise one or more flavors and or masking agents. In particular, the flavors may be natural flavors. A non-limiting example of useful flavors is vanilla.

It is important that the solid food composition is chewable. Thus, in one embodiment of the present disclosure the solid food composition has the form of a nutritional bar, a snack bar, a baked product or a combination thereof.

For example, in one embodiment of the present disclosure the solid food composition is selected from the group consisting of bread, rye bread, biscuit, tea-biscuit, cracker, pie-crust, doughnuts and combinations thereof.

In one embodiment of the present disclosure, the solid food composition does not comprise lactose.

In one embodiment of the present disclosure, the solid food composition does not comprise milk or milk derivatives.

One aspect of the present disclosure relates to a container comprising at least one solid food composition as disclosed herein, wherein the at least one solid food composition is in a package.

In one embodiment of the present disclosure the solid food composition is packaged in modified atmosphere, such as in nitrogen-enriched atmosphere.

In one embodiment of the present disclosure the package is airtight.

In one embodiment of the present disclosure the container comprises at least 7 nutritional products, such as at least 14 nutritional products, preferably at least 21 nutritional products, for example at least 28 nutritional products.

The composition may also comprise one or more drugs, e.g. any of the drugs described herein below.

Uses of the Solid Food Composition

The present inventors have found that eating the solid food of the present disclosure prior to a major meal has several beneficial effects on an individual.

In one embodiment, eating the solid food of the present disclosure prior to a major meal results in stimulation of early release of gut hormones such as GLP-1 and insulin. Consequently, these hormones will be already in circulation when the meal starts. GLP-1 will so cause a slower transit of the food through the stomach, and therefore the individual will have an increased feeling of satiety and stomach fullness. Moreover, insulin will cause glucose to be transported away from the blood more efficiently. In addition, it has been found that intake of the solid food compositions of the invention may result in a more stable blood glucose level, with lower blood glucose increase after food intake, as well as a lower drop in blood glucose levels.

One aspect of the present disclosure relates to the use of a solid food composition as disclosed herein for increasing satiety, increasing the sense of fullness, and/or reducing appetite in an individual.

One aspect of the present disclosure relates to a solid food composition as disclosed herein for use as a medicament.

One aspect of the present disclosure relates to a solid food composition as disclosed herein for use in the treatment or prevention of a metabolic disorder in an individual in need thereof.

A further aspect of the present disclosure relates to a method for treating, intervening with or preventing a metabolic disorder in an individual in need comprising administering to the individual an effective amount of the solid food composition disclosed herein.

In one embodiment of the present disclosure, the metabolic disorder is selected from the group consisting of obesity, diabetes type II, gestational diabetes mellitus, polycystic ovary syndrome (PCOS), androgen deficiency in a male individual and any combinations thereof.

One embodiment of the present disclosure relates to a solid food composition as disclosed herein for use in the treatment or prevention of a disease associated with insulin resistance in an individual in need thereof.

One embodiment of the present disclosure relates to a solid food composition as disclosed herein for use in the treatment or prevention of a disease selected from the group consisting of insulin resistance syndrome, Type 2 diabetes mellitus, impaired glucose tolerance, the metabolic syndrome, hyperglycemia, hyperinsulinemia, arteriosclerosis, hypercholesterolemia, hypertriglyceridemia, hyperlipidemia, dyslipidemia, obesity, central obesity, polycystic ovarian syndrome, microalbuminuria, hypercoagulability and hypertension and any combinations thereof, in an individual in need thereof.

One embodiment of the present disclosure relates to a solid food composition as disclosed herein for use in the treatment or prevention of gestational diabetes mellitus in an individual in need thereof.

One embodiment of the present disclosure relates to a solid food composition as disclosed herein for use in the treatment or prevention of obesity in an individual in need thereof.

One embodiment of the present disclosure relates to a solid food composition as disclosed herein for use in the treatment or prevention of diabetes type II in an individual in need thereof.

One embodiment of the present disclosure relates to a solid food composition as disclosed herein for use in the treatment or prevention of polycystic ovary syndrome (PCOS) in an individual in need thereof.

One embodiment of the present disclosure relates to a solid food composition as disclosed herein for use in the treatment or prevention of androgen deficiency in a male individual in need thereof.

One aspect of the present disclosure relates to use of the solid food composition as disclosed herein for reducing inflammation in an individual.

One aspect of the present disclosure relates to use of the solid food composition as disclosed herein for reducing blood sugar levels, blood sugar fluctuations, low-density lipoprotein (LDL) cholesterol, insulin fluctuations, and/or BMI in an individual.

One aspect of the present disclosure relates to use of the solid food composition as disclosed herein for normalizing a dysbiotic microbiota in an individual.

One aspect of the present disclosure relates to use of the solid food composition as disclosed herein for stimulating the release of incretins in the individual.

One aspect of the present disclosure relates to use of the solid food composition as disclosed herein for stimulating the release of insulin in an individual within 30 minutes after administration.

In one embodiment of the present disclosure, the solid food composition as disclosed herein reduces inflammation. Reduction of inflammation can be monitored by measuring the levels of certain parameters in blood, for example by analysing blood for presence or absence of bacterial endotoxin, and by analysing the levels of inflammatory markers such as IL-1β, IL-6, IL-10, TNF-α, C-reactive protein (CRP), and monocyte chemoattractant protein (MCP)-1.

In one embodiment of the present disclosure, the solid food composition as disclosed herein normalizes a dysbiotic microbiota. For example, in one embodiment of the present disclosure, the solid food composition as disclosed herein increases gene richness of the intestinal microbiota. For example, in one embodiment of the present disclosure, the solid food composition as disclosed herein increases the number of phylae of the intestinal microbiota. For example, in one embodiment of the present disclosure, the solid food composition as disclosed herein increases the butyrate production and/or decreases the acetate production from the intestinal microbiota. For example, in one embodiment of the present disclosure, the solid food composition as disclosed herein increases production of short chain fatty acids from the intestinal microbiota.

It is beneficial for the individual that the solid food composition as disclosed herein is eaten prior to a meal so that the food can cause the wanted response in the body prior to a meal. The solid food of the present disclosure shall be chewed and then ingested by the individual and the chewing, together with the intestinal absorption and digestion, will cause release of incretins and insulin, as well as a neural response. Generally, 15 minutes to one hour after ingestion of the solid food of the present disclosure are required to cause these responses. Thus, in one embodiment of the present disclosure, the solid food as disclosed herein is administered to an individual between one hour and 15 minutes before a meal, preferably between 45 minutes and 20 minutes before a meal, such as between 40 minutes and 30 minutes before a meal.

In one embodiment of the present disclosure, the solid food composition as disclosed herein is administered to an individual approximately 30 minutes before a meal.

It is important that the individual chews the solid food composition before ingesting it, because the action of chewing is involved in causing the wanted responses. Thus, in one embodiment of the present disclosure the solid food composition as disclosed herein, is masticated for at least 1 second, such as for at least 2 seconds, such as for at least 3 seconds, such as for at least 4 seconds, such as for at least 5 seconds, such as for at least 6 seconds, such as for at least 7 seconds, such as for at least 8 seconds, such as for at least 9 seconds, such as for at least 10 seconds.

In one embodiment of the present disclosure, the solid food composition as disclosed herein stimulates the release of incretins in said individual. Incretins are a group of metabolic hormones that stimulate a decrease in blood glucose levels, in particular, the solid food composition of the present disclosure stimulates the release of the peptides glucagon-like peptide-1 (GLP-1) and gastric inhibitory peptide (GIP). The release of incretins will cause an increase in the secretion of insulin in the individual.

Thus, in one embodiment of the present disclosure, the solid food composition as disclosed herein stimulates the release of insulin in the individual. Preferably, the solid food composition as disclosed herein stimulates the release of insulin in the individual within 30 minutes after administration.

The solid food composition of the present disclosure, thanks to the release of incretins such as GLP-1, and to the neural response caused by mastication and digestion of the solid food composition can regulate the metabolism of the individual.

Thus, in one embodiment of the present disclosure, the solid food composition as disclosed herein reduces blood sugar levels, reduces blood sugar fluctuations, reduces low-density lipoprotein (LDL) cholesterol, reduces insulin fluctuations, and/or reduces BMI of the individual.

In one embodiment of the present disclosure, the solid food composition as disclosed herein is administered to an individual at a dose in a range between 5 g and 150 g, such as at a dose in a range between 10 g and 100 g, such as at a dose in a range between 12 g and 75 g, such as at a dose in a range between 15 g and 50 g, such as at a dose of about 50 g, such as at a dose of about 20 g, such as at a dose of about 15 g.

In one embodiment of the present disclosure, the solid food composition as disclosed herein is administered to an individual daily, such as twice per day, such as three times per day.

In one embodiment of the present disclosure, the solid food composition as disclosed herein is administered to an individual for at least a week, such as for at least two weeks, such as for at least 4 weeks.

In one embodiment of the present disclosure, the solid food composition as disclosed herein is administered to an individual suffering from or suspected of suffering from a metabolic disorder. For example, in one embodiment of the present disclosure the solid food composition as disclosed herein is administered to an individual having a BMI of 25 or more, such as 30 or more, for example 35 or more, such as 40 or more. For example, in one embodiment of the present disclosure the solid food composition as disclosed herein is administered to an overweight or obese individual.

In one embodiment of the present disclosure, the solid food composition as disclosed herein is administered to an individual having a waist/hip ratio of at least 0.80, for example 0.80-0.84, such as at least 0.85 (female) or at least 0.90, for example 0.9-0.99, such as above 1.00 (male). In a further embodiment of the present disclosure, the solid food composition as disclosed herein is administered to an individual having fasting blood glucose of at least 6.1 mmol/I, for example at least 7.0 mmol/I. In an even further embodiment of the present disclosure, the solid food composition as disclosed herein is administered to an individual having a glycated haemoglobin (HbA1C) level of at least 42 mmol/mol Hb, such as between 42 and 46 mmol/mol Hb, such as at least 48 mmol/mol Hb.

In one embodiment of the present disclosure, the solid food composition as disclosed herein is administered to an individual having one or more of the following symptoms or signs:
Elevated blood pressure: ≥140/90 mmHg;
Dyslipidemia: triglycerides (TG): ≥1.695 mmol/L and high-density lipoprotein cholesterol (HDL-C) ≤0.9 mmol/L (male), ≤1.0 mmol/L (female);
Central obesity: waist:hip ratio >0.90 (male); >0.85 (female), or body mass index>30 kg/m2;
Microalbuminuria: urinary albumin excretion ratio ≥20 μg/min or albumin:creatinine ratio ≥30 mg/g;
Elevated blood glucose; and
Pathological oral glucose tolerance test (GTT).

Evaluation of blood glucose levels and of the results of GTT to establish a pathological condition is done by health providers, who know the cut-off values based on how the tests are performed and the clinical situation of the individual tested.

In one embodiment of the present disclosure, the solid food composition as disclosed herein is administered to pregnant woman.

In one embodiment of the present disclosure, the solid food composition as disclosed herein is administered to pregnant woman suffering from or suspected of suffering from a metabolic disorder.

In one embodiment the solid food composition is used in a method of reducing gastro-intestinal side effects of a drug. In such embodiments, the drug and the solid food composition are ingested either together or sequentially in any order.

Method of Manufacturing a Solid Food Composition

The invention provides methods for manufacturing solid food compositions. Said solid food compositions may be any of the solid food compositions described above in the section “Solid Food Composition”, and they may be useful for the uses described “Uses of solid food composition”.

One aspect of the present disclosure relates to a method for manufacturing a solid food composition, the method comprising:
a) Providing a protein in a range between 3 and 14% dry weight, a lipid in a range between 8 and 22% dry weight, and a complex carbohydrate in a range between 35 and 75% dry weight;
b) Treating the complex carbohydrate to reduce its phytic acid content;
c) Mixing the ingredients of a) and b) to form a slurry;
d) Heating the slurry first to a temperature in the range of between 125° C. to 140° C. for a time interval in the range of between 1 and 10 minutes,
e) Reducing then the temperature to between 70° C. and 95° C., and
f) Keeping the temperature between 70° C. and 95° C. for a time interval in the range of between 50 and 180 minutes,

thereby obtaining a solid food composition.

The methods for manufacturing the solid food composition of the invention may also comprise the steps of:
a) Providing a protein in a range between 3 and 18% dry weight, a lipid in a range between 8 and 25% dry weight, and a complex carbohydrate in a range between 35 and 75% dry weight;
b) Treating the complex carbohydrate to reduce its phytic acid content;
c) Mixing the ingredients of a) and b) to form a slurry;
d) Incubating the slurry at a high temperature in the range of between 125° C. to 140° C. for a time interval in the range of between 1 and 10 minutes; and
f) Incubating the slurry at a low temperature in the range of between 70° C. and 95° C. for a time interval in the range of between 50 and 180 minutes,
wherein steps d) and f) may be performed in any order, thereby obtaining a solid food composition.

It is preferred that the solid food compositions of the invention are baked in a manner reducing formation of Advanced Glycation End Products (AGE) as much as possible, while at the same time allowing for sufficient reduction of water content in order to obtain a dry biscuit with a long shelf life.

Thus, in order to get the lowest possible AGE, the baking should be done at low temperature for a short time. However, such conditions may be insufficient to obtain a dry biscuit.

Accordingly, it is generally preferred that the slurry is baked by incubation at a high temperature and incubation at a low temperature. It is comprised within the invention that the incubation at high temperature may be performed before or after incubation at low temperature. The incubation at high temperature, should be very short, whereas the incubation at low temperature can be longer.

In one embodiment, step e) of the method for manufacturing a solid food composition disclosed herein comprises that the temperature is reduced gradually and/or step-wise.

The method for manufacturing a solid food composition disclosed herein is characterized by being able to minimize or completely avoid the occurrence of Maillard reaction and glycation of amino acids. For obtaining this effect it is important that the temperature does not go above 140° C. It is also important that the slurry is exposed to a high temperature for a short time period, and to a lower temperature for a longer time period, as described the method of the present disclosure. In some embodiments the slurry is incubated first at high temperature and then at low temperature. Maillard reaction and glycation of amino acids is deleterious for the nutritional properties of food, in fact the adsorption of glycated amino acids by the human body is substantially reduced.

In one embodiment of the present disclosure, method for manufacturing a solid food composition disclosed herein comprises providing: a protein in a range between 3 and 18% dry weight, for example in a range between 3 and 14% dry weight, such as in a range between 5 and 14% dry weight, such as in a range between 8 and 14% dry weight, such as in a range between 10 and 14% dry weight, such as in a range between 12 and 14% dry weight, such as in a range between 3 and 12% dry weight, such as in a range between 3 and 10% dry weight, such as in a range between 3 and 8% dry weight, such as in a range between 3 and 8% dry weight, such as in a range between 3 and 5% dry weight; a lipid in a range between 8 and 25% dry weight, for example in a range between 8 and 22% dry weight, such as in a range between 8 and 20% dry weight, such as in a range between 8 and 17% dry weight, such as in a range between 8 and 15% dry weight, such as in a range between 8 and 12% dry weight, such as in a range between 8 and 10% dry weight, such as in a range between 10 and 22% dry weight, such as in a range between 12 and 22% dry weight, such as in a range between 15 and 22% dry weight, such as in a range between 17 and 22% dry weight, such as in a range between 20 and 22% dry weight; and a complex carbohydrate in a range between 35 and 75% dry weight, such as in a range between 35 and 70% dry weight, such as in a range between 35 and 65% dry weight, such as in a range between 35 and 60% dry weight, such as in a range between 35 and 55% dry weight, such as in a range between 35 and 50% dry weight, such as in a range between 35 and 45% dry weight, such as in a range between 35 and 40% dry weight, such as in a range between 40 and 75% dry weight, such as in a range between 45 and 75% dry weight, such as in a range between 50 and 75% dry weight, such as in a range between 55 and 75% dry weight, such as in a range between 60 and 75% dry weight, such as in a range between 70 and 75% dry weight; so that the sum of the protein, the complex carbohydrate and the lipid is at most 100%.

In one embodiment of the present disclosure step b) of the method of manufacturing the solid food composition disclosed herein may occur either before or after step c).

In one embodiment of the present disclosure, in the method of manufacturing the solid food composition disclosed herein step b) occurs after step c), and the method further comprises treating the complex carbohydrate and the protein to reduce their lectins content. Thus, the treatment step that reduces phytic acid content of the carbohydrate also reduces lectins content in both the carbohydrate and in the protein.

In one embodiment of the present disclosure, the method further comprises a step bb) before step c), said step comprising treating the protein to reduce its lectins content.

In one embodiment of the present disclosure the solid food composition has substantially the same nutritional composition of the slurry of c).

In one embodiment of the present disclosure, method for manufacturing a solid food composition disclosed herein comprises in step d) heating the slurry to a temperature in the range of between 125° C. to 140° C., such as to a temperature in the range of between 130° C. to 140° C., such as to a temperature in the range of between 135° C. to 140° C., for a time interval in the range of between 1 and 10 minutes, such as for a time interval in the range of between 1 and 8 minutes, such as for a time interval in the range of between 1 and 5 minutes, such as for a time interval in the range of between 1 and 3 minutes, such as for a time interval in the range of between 3 and 10 minutes, such as for a time interval in the range of between 5 and 10 minutes, such as for a time interval in the range of between 8 and 10 minutes.

In one embodiment of the present disclosure, method for manufacturing a solid food composition disclosed herein comprises in step e) thereafter reducing the temperature of the slurry to between 70° C. and 95° C. It is also comprised in the methods of the invention that step f) is performed prior to step d) in which case step e) is generally omitted.

In one embodiment of the present disclosure, method for manufacturing a solid food composition disclosed herein comprises in step e) reducing the temperature of the slurry first to between 100° C. and 120° C., such as to about 115° C., and then to between 70° C. and 95° C.

In one embodiment of the present disclosure, method for manufacturing a solid food composition disclosed herein comprises in step f) heating the slurry to a temperature in the range of between 70° C. and 95° C., such as to a temperature in the range of between 75° C. and 95° C., such as to a temperature in the range of between 80° C. and 95° C., such as to a temperature in the range of between 85° C. and 95° C., such as to a temperature in the range of between 90° C. and 95° C., such as to a temperature in the range of between 70° C. and 90° C., such as to a temperature in the range of between 70° C. and 85° C., such as to a temperature in the range of between 70° C. and 80° C., such as to a temperature in the range of between 70° C. and 75° C., for more than 1 hour, such as for a time interval in the range of between 50 and 180 minutes, such as in the range of between 55 and 180 minutes, such as in the range of between 60 and 180 minutes, such as in the range of between 65 and 180 minutes, such as in the range of between 70 and 180 minutes, such as in the range of between 75 and 180 minutes, such as in the range of between 80 and 180 minutes, such as in the range of between 85 and 180 minutes, such as in the range of between 50 and 150 minutes, such as in the range of between 50 and 120 minutes, such as in the range of between 50 and 100 minutes, such as in the range of between 50 and 90 minutes, such as in the range of between 50 and 80 minutes, such as in the range of between 50 and 75 minutes, such as in the range of between 50 and 70 minutes, such as in the range of between 50 and 65 minutes, such as in the range of between 50 and 60 minutes, such as in the range of between 50 and 55 minutes.

In one embodiment of the present disclosure, the method for manufacturing the solid food composition as disclosed herein further comprises cooling the solid food composition with sterile air.

Step b), namely treatment of the complex carbohydrate ingredient to reduce its phytic acid content, is important because phytic acid binds to and reduces absorption of minerals such as calcium, iron, and zinc. Individuals at risk of mineral deficiencies, such as vegetarians and pregnant women amongst other, should therefore preferably eat food with reduced levels of phytic acid.

In one embodiment of the present disclosure, step b) of the method for manufacturing the solid food composition as disclosed herein comprises sprouting, malting, lactic acid fermentation, enzymatic treatment, or soaking in an acid medium.

In one embodiment of the present disclosure, step b) of the method for manufacturing the solid food composition as disclosed herein comprises cold malting the complex carbohydrate.

In one embodiment of the present disclosure, step b) of the method for manufacturing the solid food composition as disclosed herein comprises fermentation by steeping. For example, an effective time-temperature matrix is used such that it is capable of activating phytase, such as phytase naturally present in the solid food and/or complex carbohydrate ingredient, and being not damaging for the β-glucans molecular weight profile.

In one embodiment of the present disclosure, step b) of the method for manufacturing the solid food composition as disclosed herein comprises treating the complex carbohydrate and/or a β-glucan concentrate with phytase enzymes.

Additionally, in one embodiment of the present disclosure, the method for manufacturing the solid food composition as disclosed herein reduces both phytic acid and lectins content of the least one complex carbohydrate ingredient. This may be achieved by soaking the complex carbohydrate (which in some embodiments is oat) in water or, alternatively, in the slurry of step c), for 5 to 12 hours, such as for 8 to 12 hours, such as for 10 to 12 hours, such as for 5 to 10 hours, such as for 5 to 8 hours, at a temperature of 8° C. to 25° C., such as at a temperature of 10° C. to 25° C., such as at a temperature of 13° C. to 25° C., such as at a temperature of 15° C. to 25° C., such as at a temperature of 18° C. to 25° C., such as at a temperature of 20° C. to 25° C., such as at a temperature of 22° C. to 25° C., such as at a temperature of 8° C. to 22° C., such as at a temperature of 8° C. to 20° C., such as at a temperature of 8° C. to 18° C., such as at a temperature of 8° C. to 15° C., such as at a temperature of 8° C. to 13° C., such as at a temperature of 8° C. to 10° C.

In one embodiment of the present disclosure the method for manufacturing the solid food composition as disclosed herein reduces both phytic acid and lectins content of the least one complex carbohydrate ingredient and of the at least one protein ingredient. This may be achieved by soaking the complex carbohydrate and the protein ingredients in the slurry of step c), for 5 to 12 hours, such as for 8 to 12 hours, such as for 10 to 12 hours, such as for 5 to 10 hours, such as for 5 to 8 hours, at a temperature of 8° C. to 25° C., such as at a temperature of 10° C. to 25° C., such as at a temperature of 13° C. to 25° C., such as at a temperature of 15° C. to 25° C., such as at a temperature of 18° C. to 25° C., such as at a temperature of 20° C. to 25° C., such as at a temperature of 22° C. to 25° C., such as at a temperature of 8° C. to 22° C., such as at a temperature of 8° C. to 20° C., such as at a temperature of 8° C. to 18° C., such as at a temperature of 8° C. to 15° C., such as at a temperature of 8° C. to 13° C., such as at a temperature of 8° C. to 10° C. Thus, in some embodiments, step c) occurs prior to the step b) of treating the at least one complex carbohydrate ingredient to reduce its phytic acid content and further comprises treating the at least one complex carbohydrate ingredient and the at least one protein ingredient to reduce their lectins content.

The phytic acid reduction following these procedures can be in the range of 5 to 30-fold, such as at least 10-fold, for example at least 20-fold, such as 25-fold. In particular, the methods may result in a reduction of the level of phytic acid to less than 70%, preferably to less than 60%, such as to less than 50% of the initial level. In embodiments of the invention, where the solid food composition comprises oat grains as the main ingredient, preferably, said oat ingredients are treated to reduce the level of phytic acid to less than less than 0.7 g, preferably to less than 0.6 g, such as to less than 0.5 g per 100 g dry weight of said oat grains.

Several methods can be used to analyse the phytic acid content of a food ingredient e.g. spectrophotometry or HPLC. For example phytic acid may be measured by first extracting it and precipitating it as ferric phytic acid (Wheeler & Ferrel, 1971), followed bydetrmining the iron content by Makower's method (1970).

Several methods can be used to analyse the lectins content of a food ingredient e.g. spectrophotometry or HPLC. ELISA kits for measurements of Individual lectins can be obtained from several companies e.g. Abcam (Cambridge, UK) and Aviva Systems Biology (San Diego, Calif., USA). A micro array based screening technology has been described by Kletter et al. (2013).

In one embodiment of the present disclosure, the method for manufacturing the solid food composition as disclosed herein further comprises a step of grinding the solid food composition to form a granulated product. The so formed granulated product may be eaten as such or stored and used successively to manufacture a solid food composition, which has substantially the same nutritional value as the solid food composition prior to grinding.

Additional Methods of Manufacturing a Solid Food Composition

The invention provides additional methods for manufacturing solid food compositions. Said solid food compositions may be any of the solid food compositions described above in the section “Solid Food Composition”, and they may be useful for the uses described “Uses of solid food composition”.

The additional methods for manufacturing the solid food compositions of the invention may comprise the steps of:
A. Providing cereal grains;
B. Treating said grains to reduce their phytic acid content;
C. Subjecting said grains to a step of heat treatment;
D. Finely dividing said grains
E. Adding at least one liquid and one or more additional ingredients to said finely divided grains to obtain a slurry
F. Incubating the slurry at a low temperature in the range of between 60° C. and 95° C. for a time interval in the range of between 30 and 180 minutes; and
G. Incubating the slurry at a high temperature in the range of between 125° C. to 140° C. for a time interval in the range of between 1 and 10 minutes;
wherein step B. may be performed at any time during the method and steps F. and G. may be performed in any order, thereby obtaining a solid food composition.

The cereal grains to be used with the methods of the invention are preferably oat grains and more preferably dehulled oat grains. Oat grains comprises high levels of complex carbohydrates, such as beta-glucans as well as proteins, and are thus particularly suitable as an ingredient for the solid food compositions of the invention.

Oat further comprises lectins and phytic acid. The inventors have found that a low level of phytic acid is beneficial for Preload compositions. Also low levels of lectin has been found to be beneficial for Preload compositions.

Accordingly, it is preferred that said cereal grains (oat grains) have been treated to reduce the level of phytic acid. Preferably, said step treatment of phytic acid also results in reduction of lectins, whereas the treatment preferably is performed in a manner preserving as many complex carbohydrates (e.g. beta-glucans) as possible.

In a preferred embodiment, the step of treating said (oat) grains to reduce their phytic acid content comprises or even consists of malting said (oat) grains.

Malting is a process where cereal grains are germinated under controlled environmental conditions. Thus, said step of treating said (oat) grains to reduce their phytic acid content may comprise the steps of
B1. submerging said (oat) grains in water
B2. germinating said (oat) grains.

The step of submerging said oat grains in water may also be referred to as “soaking”. In general soaking is performed in a manner, where the (oat) grains are completely submerged in water.

It has been found that soaking (oat) grains in water results in reduction of both phytic acid and lectins in said grains. Whereas longer soaking times typically leads to a higher reduction in phytic acid, longer soaking times may also lead to an unwanted reduction in beta-glucan levels. In one embodiment of the invention, step B1. comprises or consists of submerging said (oat) grains in water for in the range of 2 to 24 hours, such as for in the range of 2 to 12 hours, for example for in the range of 3 to 10 hours, such as in the range of to 4 to 8 hours, such as for in the range of 5 to 7 hours, for example for approx. 6 hours.

The step of submerging said (oat) grains may be performed at any useful temperature, preferably said (oat) grains may be submerged in water at a temperature of in the range of 20 to 30° C., such as in the range of 21 to 27° C., for example in the range of 23 to 25° C.

Once the grains have been soaked, the grains may be germinated. Typically, germination involves incubation of the soaked grains in air at ambient temperature. In particular, the (oat) grains may be allowed to germinate for in the range of 10 to 80 hrs, for example for in the range of 10 to 40 hrs, such as in the range of 15 to 35 hrs. for example for in the range of 20 to 25 hrs.

The treatment to reduce the level of phytic acid may also involve adding phytase. It is comprised within the invention that the treatment to reduce the level of phytic acid may involve both malting of the (oat) grains and addition of phytase. It is also comprised within the invention that said treatment to reduce the level of phytic acid consists of adding phytase. Said phytase may be added to the solid food composition at any useful time, however frequently phytase is added at the same time as the other additional ingredients are added, i.e. during step E. Thus steps B. and E. may be performed simultaneously or partly simultaneously.

Said phytase may be any phytase, e.g. any type of phosphatase enzyme that catalyzes the hydrolysis of phytic acid (myo-inositol hexakisphosphate)

The treatment to reduce the level of phytic acid is preferably performed in a manner reducing the level of phytic acid in said (oat) grains to less than 70%, preferably to less than 60%, such as to less than 50% of the initial level.

Oat grains typically comprises in the range of 1 to 1.3 g phytic acid per 100 g grains (dry weight). It is preferred that the grains comprise less than 0.7 g, preferably to less than 0.6 g, such as to less than 0.5 g phytic acid per 100 g dry weight of said grains after completion of step B. This is in particular the case in embodiments of the invention, where the grains are oat grains.

High lipase activity in the (oat) grains is generally less preferred, because it can lead to rancid taste and shorter shelf life.

Preferably the methods of the invention comprise a step of heat treatment. The inventors have found that heat treatment of germinated (oat) grains significantly reduces lipase activity. It is preferred that the heat treatment is performed in a manner reducing lipase activity by at least 50%, such as reducing lipase activity by at least 70%.

This may for example be achieved by heating the grains, e.g. by incubating (oat) grains at a temperature in the range of 90 to 120°, such as in the range of 95 to 100°. Said incubation may e.g. be performed for in the range of 30 to 600 min, such as in the range of 60 to 120 min.

Alternatively, this may for example be achieved by microwave treatment of said (oat) grains. Said microwave treatment may for example be performed by subjecting the oat grains to microwaves at in the range of 800 to 1400 W, such as in the range of 900 to 1200 W. Said microwaving may for example be performed for in the range of 30 to 120 s, such as in the range of 40 to 60 s.

Once the (oat) grains have been subjected to heat treatment, the grains are typically finely divided, which in general results in a flour. The grains may be finely divided by any useful method, e.g. by blending or grinding or milling.

Step E. of the method may comprise addition of a liquid and additional ingredients. Said liquid may in particular be water. Typically, water is added to the finely divided grains in an amount allowing formation of a slurry. For example, the weight of water added may be in the range of 0.3 to 3 times dry weight of the grains.

The additional ingredients may be any of the ingredients described herein above in the section “Solid food composition”. The additional ingredients may for example be one or more of:
Salt;
soluble fibers, e.g. beta-glucans, e.g. any of the beta-glucans described above in the section “Solid food composition”;
sweeteners, e.g. any of the sweeteners described above in the section “Solid food composition”;
natural flavors, e.g. any of the natural flavors described above in the section “Solid food composition”;
Lipids, e.g. vegetable oils, such as any of the vegetable oils described above in the section “Solid food composition”.

A slurry is prepared by mixing the finely divided (oat) grains, liquid and additional ingredients. The slurry may be formed into any desired shape, e.g. using a mould, and is then baked. As described above in the section “Method of manufacturing a solid food composition” it is preferred that the solid food compositions of the invention are baked in a manner reducing formation of Advanced Glycation End Products (AGE) as much as possible, while at the same time allowing for sufficient reduction of water content in order to obtain a dry biscuit with a long shelf life. As also described above, it is preferred that the methods minimize or completely avoid the occurrence of Maillard reaction and glycation of amino acids.

Thus as described above in the section “Method of manufacturing a solid food composition”, the slurry is baked at relatively low temperatures. Typically, the baking is performed in a two-step method comprising a short incubation at a high temperature and a long incubation at a low temperature. These incubations can be performed in any order.

The incubation a low temperature may be an incubation at in the range of between 60° C. and 95° C., such as at a temperature in the range of 70 to 90° C. In one embodiment the incubation at low temperature is performed at a temperature in the range of 65 to 75° C., such as in the range of 68 to 72° C.

Said incubation at low temperature may for example be performed for in the range of between 30 and 180 minutes, for example for in the range of 50 to 180 min, such as in the range of 50 to 90 min.

The incubation at high temperature may for example be an incubation at a temperature in the range of between 125° C. to 140° C.

Said incubation at high temperature may for example be performed for in the range of between 1 and 10 minutes, such as in the range of 5 to 9 min.

In addition to aforementioned additional ingredients, a drug may also be added to the solid food compositions. Typically, said drug will be added during step E. The drug may e.g. be any of the drugs described below in the section “Drug”.

Drug

The compositions of the invention may in addition to the compounds described above, also comprise one or more active ingredients, for example one or more drugs. Alternatively, the compositions of the invention and a drug may be administered separately to an individual in need thereof. Thus, the invention also provides kits-of-part comprising the compositions of the invention and one or more drugs.

Regardless of whether the drug is comprised in the compositions of the invention or administered separately to said compositions, the aim is typically to reduced gastro-intestinal side effects of said drug. Thus, the drug may for example be any drug having gastro-intestinal side effects. For example, the drug may be any of the drugs causing gastrointestinal side effects described in Jian et al. 2009, e.g, any of the drugs listed in Tables 1, 5 or 6 therein.

In one embodiment, the drug may be a bile acid sequesters, for example cholestyramine.

In one embodiment, the drug may be an anti-inflammatory drug or an analgesic drugs, for example a drug selected from the group consisting of aspirin, NSAID and opioids.

In one embodiment, the drug may be an antibiotic, for example cephalosporins or penicillins.

In one embodiment, the drug may be an antiviral drug, for example Tamiflu or Avigan.

In one embodiment, the drug may be a drug used for parasite infestations for example Mebendazole.

In one embodiment, the drug may be a neurologically acting drug, for example Prozac.

The individual to be treated with a combination of the compositions of the invention and a drug may be any animal, for example humans or domestic animals.

Items

The invention may further be defined by any one of the following items:
1. A solid food composition comprising:
   a protein in a range between 3 and 18% dry weight,
   a lipid in a range between 8 and 25% dry weight,
   a complex carbohydrate in a range between 35 and 80% dry weight,
   soluble fibers in a range between 2 and 19% dry weight
wherein the complex carbohydrate has been treated to comprise a reduced amount of phytic acid compared to the untreated carbohydrate, and wherein all ingredients of the composition are of plant origin.
2. The composition according to item 1, wherein the composition comprises a protein in a range between 3 and 14% dry weight.
3. The composition according to any one of the preceding items, wherein the composition comprises a lipid in a range between 8 and 22% dry weight.
4. The composition according to any one of the preceding items, wherein the complex carbohydrate has been treated to comprise a reduced amount of lectins compared to the untreated carbohydrate.
5. The composition according to any one of the preceding items, wherein the protein has been treated to comprise a reduced amount of lectins compared to the untreated protein.
6. The composition according to any one of the preceding items, wherein said treatment is a heat treatment.
7. The composition according to any one of the preceding items, wherein said composition comprises a low level of phytic acid.
8. The composition according to any one of the preceding items, wherein said composition has a level of phytic acid below 5‰, preferably below 4‰, such as below 3‰.
9. The composition according to any one of the preceding items, wherein said composition comprises a low level of lectins.
10. The composition according to any one of the preceding items, wherein the protein is protein isolate obtained from a tuber, a seed or a legume.
11. The composition according to any one of the preceding items, wherein the protein is protein isolate obtained from potatoes, oat, hemp, peas, beans, lentils, soy, quinoa, amaranth, breadnut, chia, kaniwa, spirulina and nuts.
12. The composition according to any one of the preceding items, wherein the protein is protein isolate obtained from potatoes, oat or hemp.
13. The composition according to any one of the preceding items, wherein the protein is comprised in oat grains, and the composition comprises said grains or parts thereof.
14. The composition according to any one of the preceding items, wherein the lipid is a vegetable oil.
15. The composition according to any one of the preceding items, wherein the lipid is selected from a group consisting of coconut oil, sunflower oil, rapeseed oil, canola oil, peanut oil, corn oil, palm oil, avocado oil, walnut oil, brassica oil, olive oil and linseed oil.
16. The composition according to any one of the preceding items, wherein the complex carbohydrate comprises β-glucans.
17. The composition according to any one of the preceding items, wherein the complex carbohydrate is comprised in grains of a cereal or a pseudocereal, and the composition comprises said grains or parts thereof.
18. The composition according to any one of the preceding items, wherein the complex carbohydrate is comprised in grains of a gluten free cereal or pseudocereal.
19. The composition according to any one of the preceding items, wherein the complex carbohydrate is comprised in grains of a gluten free cereal or pseudocereal selected from a group consisting of oat, corn, rice, millets and buckwheat, and the composition comprises said grains or parts thereof.
20. The composition according to any one of the preceding items, wherein the complex carbohydrate is comprised in oat grains, and the composition comprises said grains or parts thereof.
21. The composition according to any one of the preceding items, comprising protein isolate from potatoes (Solanum tuberosum) in a range between 4 to 8% dry weight, coconut oil in a range between 12 to 18% dry weight, and oat in a range between 55 to 70% dry weight.
22. The composition according to any one of the preceding items, comprising oat in a range between 55 to 70% dry weight, coconut oil in a range between 12 to 18% dry weight and β-glucans concentrate in a range between 10 and 20% by weight.
23. The composition according to item 22, wherein the β-glucans concentrate comprises soluble and insoluble fibers, and wherein the soluble fibers are at least 20% by weight of the β-glucans concentrate.
24. The composition according to any one of the preceding items, wherein the soluble fibers of the β-glucans concentrate comprise at least 20% by weight of high molecular weight β-glucans, such as at least 25% by weight of high molecular weight β-glucans.
25. The composition according to any one of the preceding items, wherein the high molecular weight β-glucans have a weight average molecular weight of 30.000 g/mol or higher, such as of 50.000 g/mol or higher.
26. The composition according to any one of the preceding items, wherein the β-glucans concentrate comprises at least 50% by weight insoluble fibers.
27. The composition according to any one of the preceding items, wherein the solid food composition has a glycemic index below 55.
28. The composition according to any one of the preceding items, further comprising water soluble vitamins and/or lipid soluble vitamins and/or minerals and/or additional amino acids.
29. The composition according to any one of the preceding items, further comprising a sweetener and/or one or more natural flavors.
30. The composition according to any one of the preceding items, wherein the sweetener is agave syrup.
31. The composition according to any one of the preceding items, wherein the composition has the form of a nutritional bar, a snack bar, a baked product or a combination thereof.
32. The composition according to any one of the preceding items, wherein the composition is selected from the group consisting of bread, rye bread, biscuit, tea-biscuit, cracker, pie-crust, doughnuts, granulate and combinations thereof.
33. Use of a solid food composition according to any one of the preceding items for increasing satiety, increasing the sense of fullness, and/or reducing appetite.
34. A solid food composition according to any one of the preceding items for use as a medicament.
35. A solid food composition according to any one of the preceding items for use in the treatment or prevention of a metabolic disorder.
36. The solid food composition according to any one of the preceding items for use in a method of reducing inflammation in an individual.
37. The solid food composition according to any one of the preceding items for use in a method of reducing blood sugar levels, blood sugar fluctuations, low-density lipoprotein (LDL) cholesterol, insulin fluctuations, and/or BMI in an individual.
38. The solid food composition according to any one of the preceding items for use in a method of normalizing a dysbiotic microbiota in an individual.
39. The solid food composition according to any one of the preceding items for use in a method of stimulating the release of incretins in the individual.
40. The solid food composition according to any one of the preceding items for use in a method of stimulating the release of insulin in an individual within 30 minutes after administration.
41. Use of the solid food composition according to any one of the preceding claims for reducing inflammation in an individual.
42. Use of the solid food composition according to any one of the preceding claims for reducing blood sugar levels, blood sugar fluctuations, low-density lipoprotein (LDL) cholesterol, insulin fluctuations, and/or BMI in an individual.
43. Use of the solid food composition according to any one of the preceding claims for normalizing a dysbiotic microbiota in an individual.
44. Use of the solid food composition according to any one of the preceding claims for stimulating the release of incretins in the individual.
45. Use of the solid food composition according to any one of the preceding claims for stimulating the release of insulin in an individual within 30 minutes after administration.
46. The composition for use or the use according to any one of items 35 to 45, wherein the solid food composition is administered to an individual between one hour and 15 minutes before a meal, preferably between 45 minutes and 20 minutes before a meal, such as between 40 minutes and 30 minutes before a meal.
47. The composition for use or the use according to any one of items 35 to 46, wherein the solid food composition is administered to an individual at a dose in a range between 5 g and 150 g, such as at a dose in a range between 10 g and 100 g, such as at a dose in a range between 12 g and 75 g, such as at a dose in a range between 15 g and 50 g, for example at a dose in the range of 15 to 30 g, such as at a dose of about 50 g, such as at a dose of about 27 g, for example at a dose about 20 g, such as a dose about 18 g, such as at a dose of about 15 g.
48. The composition for use or the use according to any one of items 35 to 47, wherein the solid food composition is administered to an individual suffering from or suspected of suffering from a metabolic disorder daily, such as twice per day, such as three times per day.
49. The composition for use or the use according to any one of items 35 to 48, wherein the solid food composition is administered to an individual suffering from or suspected of suffering from a metabolic disorder for at least a week, such as for at least two weeks, such as for at least 4 weeks.
50. The composition for use or the use according to any one of items 35 to 49, wherein the individual has a BMI of 25 or more, such as 30 or more, for example 35 or more, such as 40 or more.
51. The composition for use according to any one of items 35 to 50, wherein the individual is overweight or obese.
52. The composition for use or the use according to any one of items 35 to 51, wherein the solid food composition is administered to an individual suffering from or suspected of suffering from a metabolic disorder.
53. The composition for use or the use according to any one of items 35 to 52, wherein the solid food composition is administered to an individual suffering from or suspected of suffering from elevated blood sugar.
54. The composition for use or the use according to any one of items 35 to 53, wherein the individual is a pregnant woman.
55. The composition for use or the use according to any one of items 35 to 54, wherein the metabolic disorder is selected from the group consisting of obesity, diabetes type II, gestational diabetes mellitus, polycystic ovary syndrome (PCOS), androgen deficiency in a male individual and any combinations thereof.
56. A method for treating or preventing a metabolic disorder in an individual comprising administering to the individual an effective amount of the solid food composition according to any one of items 1 to 32.
57. A method for manufacturing a solid food composition, the method comprising:
   a) Providing a protein in a range between 3 and 14% dry weight, a lipid in a range between 8 and 22% dry weight, and a complex carbohydrate in a range between 35 and 75% dry weight;
   b) Treating the complex carbohydrate to reduce its phytic acid content;
   c) Mixing the ingredients of a) and b) to form a slurry;
   d) Heating the slurry first to a high temperature in the range of between 125° C. to 140° C. for a time interval in the range of between 1 and 10 minutes; and
   e) Reducing then the temperature to between 70° C. and 95° C., and
   f) Keeping the low temperature between 70° C. and 95° C. for a time interval in the range of between 50 and 180 minutes,
   thereby obtaining a solid food composition.
58. A method for manufacturing a solid food composition, the method comprising:
   a) Providing a protein in a range between 3 and 18% dry weight, a lipid in a range between 8 and 25% dry weight, and a complex carbohydrate in a range between 35 and 75% dry weight;
   b) Treating the complex carbohydrate to reduce its phytic acid content;
   c) Mixing the ingredients of a) and b) to form a slurry;
   d) Incubating the slurry at a high temperature in the range of between 125° C. to 140° C. for a time interval in the range of between 1 and 10 minutes; and
f) Incubating the slurry at a low temperature in the range of between 60° C. and 95° C. for a time interval in the range of between 30 and 180 minutes, wherein steps d) and f) may be performed in any order, thereby obtaining a solid food composition.
59. The method according to item 57, wherein step b) may occur before or after step c).
60. The method according to any one of items 57 to 59, wherein step b) occurs after step c), and wherein the method further comprises treating the at least one complex carbohydrate and/or the at least one protein to reduce their lectins content.
61. The method according to any one of items 57 to 60, wherein the solid food composition has substantially the same nutritional composition of the slurry of c).
62. The method according to any one of items 57 to 61, further comprising cooling the composition with sterile air.
63. The method according to any one of items 57 to 62, wherein step b) comprises sprouting, malting, lactic acid fermentation, enzymatic treatment, or soaking in an acid medium, for example treatment with phytase.
64. The method according to any one of items 57 to 63, wherein step b) comprises cold malting the complex carbohydrate.
65. The method according to any one of items 57 to 64, wherein said complex carbohydrate is comprised within oat grains, and wherein step b) comprises or consists of cold malting said oat grains.
66. A method for manufacturing a solid food composition, the method comprising:
   A. Providing cereal grains;
   B. Treating said grains to reduce their phytic acid content;
   C. Subjecting said grains to a step of heat treatment;
   D. Finely dividing said grains
   E. Adding at least one liquid and one or more additional ingredients to said finely divided grains to obtain a slurry
   F. Incubating the slurry at a low temperature in the range of between 60° C. and 95° C. for a time interval in the range of between 30 and 180 minutes; and
   G. Incubating the slurry at a high temperature in the range of between 125° C. to 140° C. for a time interval in the range of between 1 and 10 minutes;
   wherein step B. may be performed at any time during the method and steps F. and G. may be performed in any order, thereby obtaining a solid food composition.
67. The method according to item 66, wherein the cereal grains are dehulled oat grains.
68. The method according to any one of items 66 to 67, wherein step B. comprises or consists of malting said grains.
69. The method according to any one of items 66 to 68, wherein step B. comprises the steps of
   B1. submerging said grains in water
   B2. germinating said grains.
70. The method according to item 69, wherein step B1) comprises or consists of submerging said oat grains in water for in the range of 2 to 24 hours, such as for in the range of 2 to 12 hours, for example for in the range of 3 to 10 hours.
71. The method according to any one of items 69 to 70, wherein step B1) comprises or consists of submerging said grains in water for in the range of 4 to 8 hours, such as for in the range of 5 to 7 hours, for example for approx. 6 hours.
72. The method according to any one of items 69 to 71, wherein step B1. comprises or consists of submerging said grains in water at a temperature of in the range of 20 to 30° C., such as in the range of 21 to 27° C., for example in the range of 23 to 25° C.
73. The method according to any one of items 69 to 72, wherein step B2. comprises or consists of germinating said grains for in the range of 10 to 80 hrs, for example for in the range of 10 to 40 hrs, such as in the range of 15 to 35 hrs. for example for in the range of 20 to 24 hrs.
74. The method according to any one of items 66 to 73, wherein step B. comprises addition of phytase.
75. The method according to any one of items 66 to 74, wherein step B. is performed in a manner reducing the level of phytic acid in said grains to less than 70%, preferably to less than 60%, such as to less than 50% of the initial level.
76. The method according to any one of items 66 to 75, wherein the cereal grains comprise less than 0.7 g, preferably to less than 0.6 g, such as to less than 0.5 g phytic acid per 100 g dry weight of said grains after completion of step B.
77. The method according to any one of items 66 to 76, wherein step B. is performed simultaneously with step e).
78. The method according to any one of items 66 to 77, wherein step C. is performed in a manner reducing lipase activity by at least 50%, such as reducing lipase activity by at least 70%.
79. The method according to any one of items 66 to 78, wherein step C. is performed by incubating grains at a temperature in the range of 90 to 120°, such as in the range of 95 to 100°.
80. The method according to item 79, wherein said incubation is performed for in the range of 30 to 600 min, such as in the range of 60 to 120 min.
81. The method according to any one of items 66 to 80, wherein step C. is performed by subjecting the grains to microwaves at in the range of 800 to 1400 W, such as in the range of 900 to 1200 W.
82. The method according to item 81, wherein said incubation is performed for in the range of 30 to 120 s, such as in the range of 40 to 60 s.
83. The method according to any one of items 66 to 82, wherein said liquid added in step E. is water.
84. The method according to any one of items 66 to 83, wherein one additional ingredient is salt.
85. The method according to any one of items 66 to 84, wherein one additional ingredient comprises a soluble fiber.
86. The method according to any one of items 66 to 85, wherein one additional ingredient is beta-glucan, such as beta-glucan as defined in any one of items 23 to 26.
87. The method according to any one of items 66 to 86, wherein one or more additional ingredients are sweeteners and/or natural flavors.
88. The method according to any one of items 66 to 87, wherein one additional ingredient is a lipid, for example a lipid as defined in any one of items 14 to 15.
89. The method according to any one of items 57 to 88, wherein the step of incubating the slurry at a low temperature is performed a temperature in the range of 70 to 90° C.
90. The method according to any one of items 57 to 89, wherein the step of incubating the slurry at a low temperature is performed a temperature in the range of 65 to 75° C., such as in the range of 68 to 72° C.
91. The method according to any one of items 57 to 90, wherein the step of incubating the slurry at a low temperature is performed for in the range of 50 to 180 min, such as in the range of 50 to 90 min.
92. The method according to any one of items 57 to 91, wherein the method comprises a further step of grinding the solid food to form a granulated product.
93. The method according to any one of items 57 to 92, wherein the method comprises a further step of adding a drug.
94. The method according to item 93, wherein said drug compound is a drug having gastro-intestinal side effects.
95. The method according to any one of items 93 to 94, wherein said drug is selected from the group consisting of bile acid sequesters, anti-inflammatory drugs, analgesics, antibiotics, anti-viral drugs and neurologically acting drugs.
96. A solid food composition manufactured by the method according to any one of items 57 to 95.
97. The solid food composition according to any one of items 1 to 32, wherein the composition has been prepared by the method according to any one of items 57 to 95.
98. The composition according to any one of items 1 to 32 and 96 to 97, wherein the composition further comprises a drug compound.
99. A kit-of-parts comprising
   A. the compositions according to any one of items 1 to 32 and 96 to 98; and
   B. a drug.
100. The composition or kit-of-parts according to any one of items 98 to 99, wherein said drug compound is a drug having gastro-intestinal side effects.
101. The composition or kit-of-parts according to any one of items 98 to 100, wherein said drug is selected from the group consisting of bile acid sequesters, anti-inflammatory drugs, analgesics, antibiotics, anti-viral drugs and neurologically acting drugs.
102. A solid food composition according to any one of items 96 to 97 for use in a method of treatment or prevention of a metabolic disorder.
103. The composition for use according to item 102, wherein said use is as specified in any one of items 33 to 55.
104. A container comprising at least one solid food composition as defined in any one of the preceding items, wherein the at least one solid food composition is in a package.
105. The container according to item 103, wherein solid food composition is packaged in modified atmosphere, such as in nitrogen-enriched atmosphere.
106. The container according to any one of items 104 and 105, wherein the package is airtight.
107. The container according to any one of items 104 to 106, wherein the container comprises at least 7 nutritional products, such as at least 14 nutritional products, preferably at least 21 nutritional products, for example at least 28 nutritional products.

### EXAMPLES

### Example 1. Composition of an Optimized Preload

Non-animal based ingredients were used to prepare the composition described below. Potatoes (Solanum tuberosum) in the form of dry powder isolate was mixed together with coconut oil and a water soaked oat (cold malting; done to degrade phytic acid naturally present in oats) according to the following manufacturing process:
Allow the oat soak in cold water in chilled temperature (circa 6-8° C.) for at least 4 hours;
Add Agave syrup and coconut oil and stir into a slurry;
Mix all other dry components separately and add into the slurry and mix into a sticky dough.

The weight of each component and its percentage of the composition is given in Table 1.

TABLE 1
Example of a composition of a preload biscuit

| Ingredient | Dry weight gr (% dry weight) |
|---|---|
| Potatoes protein | 100 gr (6%) |
| Coconut oil | 250 gr (15%) |
| Oat | 1100 gr (66%) |
| Agave syrup | 150 gr (9%) |
| Salt | 10 gr (0.5%) |
| Bicarbonate | 50 gr (3%) |
| Vanilla | 10 gr (3%) |

TABLE 2
Example of a composition of a preload biscuit

| Ingredient | Dry weight gr (% dry weight) |
|---|---|
| De-hulled oats | 1350 gr (58%) |
| Coconut oil | 375 gr (16%) |
| Dry fractionated Beta-Glucan | 350 gr (15%) |
| Agave syrup | 225 gr (10%) |
| Salt | 15 gr (0.5%) |
| Vanilla | 15 gr (0.5%) |

The measurement of phytic acid reduction of one preferred form of preload is shown in Table 3. The preload composition of Table 1 was soaked in water and incubated at 20° C. for 12 hours. Following extraction, phytic acid was measured and the data without oat soaking was set to 100%. It was found that the soaking treatment reduced phytic acid content by 92%, as shown in Table 3. Further duration of soaking and the continued process can remove final residues of phytic acid.

TABLE 3
Measurement of phytic acid in preload with and
without soaking treatment.

| Phytic acid (%)-No soaking | Phytic acid (%)-After soaking |
|---|---|
| 100% | 8% |

Moreover, the β-glucans content in preload was of between 7 and 8% w/w, as measured by AOAC Official Method 995.16, which is normally used for measuring β-glucans in cereals.

Conclusion: The non-animal based solid food composition was gluten- and lactose-free, and was characterized by a low phytic acid content, in particular a 92% lower phytic acid content compared to a composition comprising untreated oat.

### Example 2. Baking Process for Production of an Optimized Preload with a High Nutritional Value

To avoid elevated Maillard reactions in the product causing formation of glycated amino acids, a longer baking process at a lower temperature is required to stabilize the end product. Hence, the product was baked in 135° C. for only 3 minutes, such as for up to 10 minutes, and then the temperature was lowered to 90° and kept there for at least 1 hour to reduce water activity and stabilize the product. It is important not to reach temperatures higher than 140° C., which would cause Maillard reaction to occur with the consequent glycation of the amino acid residues present in the product. Moreover, it is important to notice that the temperature of the product reached at the most 120° C., hence not causing glycation to be induced.

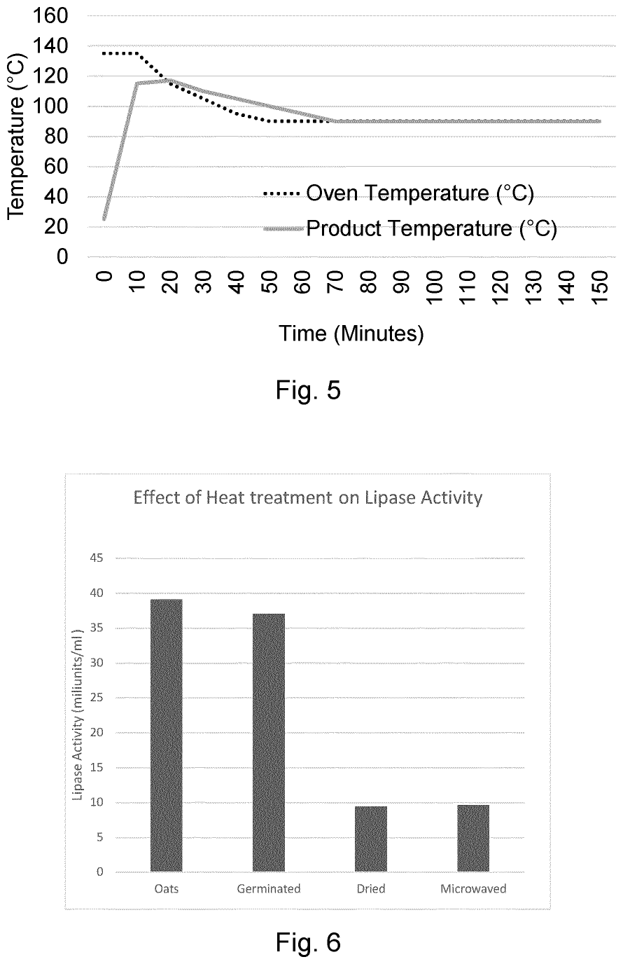

The temperature was for example lowered to 115° C. at 20 minutes, and then to 95° C. at 40 minutes, and then to 90° C. at 50 minutes. The temperature was then kept at 90° C. for more than 1 hour (see FIG. 5).

Preload composed of the ingredients described in Example 1 was exposed to two different baking procedures:

a) conventional baking (15 min at 200° C.); and

b) baking according to the present disclosure, as described above in this example.

The glycation of proteins (% of glycated amino acids) were estimated by using data in the literature. The actual glycation was determined using mass spectrometry. The results as a percentage are shown in Table 4.

TABLE 4
Formation of glycated amino acid residues due to baking

| Conventional baking | | Baking according to the | |
|---|---|---|---|
| procedure | | present disclosure | |
| Estimated | Measured | Estimated | Measured |
| glycation* | glycation* | glycation* | glycation* |
| 100% | 100% | 15% | 10-53% |

*% of glycated amino acids.

The amount of estimated and measured glycated amino acids was set to 100, the estimated and measured glycated amino acids is expressed as percentage of the value obtained for the conventional baking procedure.

The product was cool down with sterile air and then packed in airtight packaging with modified atmosphere (nitrogen). Due to the low water activity and modified atmosphere the product reached a shelf life of 2 years with maintained nutritional values.

Conclusion: The non-animal based solid food composition was gluten- and lactose-free, and was characterized by a strongly reduced content of glycated amino acid residues compared to the same composition baked according to a conventional baking procedure.

### Example 3. Packaging of Preload and Creation of a Mobile Phone Connected Application

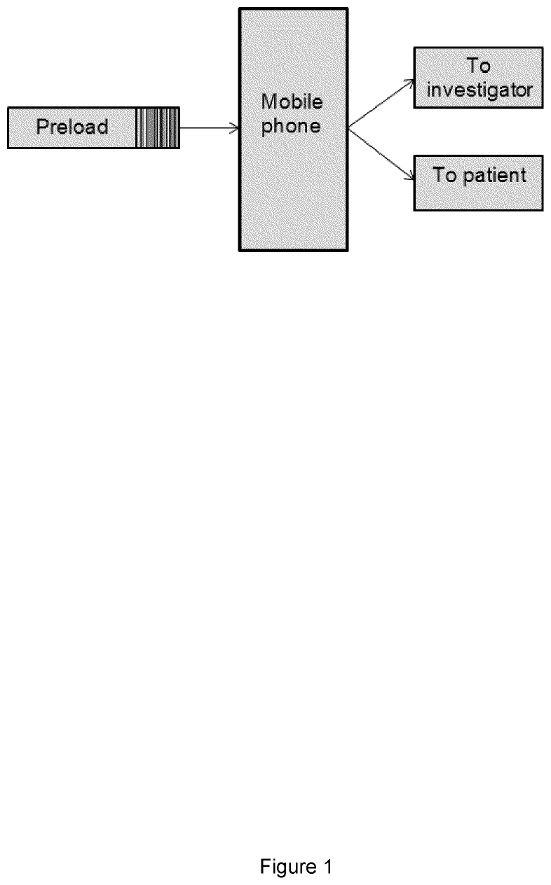

The optimized preload product is packaged into 21 biscuits sufficing for one week treatment. The package is provided with a bar code and/or QR code which can be read with a mobile phone. The reading of the bar code sets a time for the start-time of the treatment and so it can provide reminder to the subject, for example daily, to ingest the preload biscuit, and also report when the package has to be refilled or substituted. The mobile phone application can in addition provide the treated subject with relevant advice and information on gestational diabetes (GDM) and it can also be connected to continuous glucose recordings. Optionally, the mobile application can contain an element of reward if instructions have been followed. An outline of the App is given in FIG. 1. In addition, the mobile phone application provides information to the investigator, such as when the treatment has started, glucose reading eventually taken, and other relevant information.

### Example 4. Effect of Preload on Blood Parameters

A. Effect on rapid read-outs. Preload according to Examples 1-2 is given to subjects to evaluate effects on GLP-1. Healthy volunteers are given Preload in the morning (before breakfast) and blood samples are collected with 10 min intervals up till 45 min. GLP-1 is measured using a commercial kit for immunodetection of Glucagon-like peptide 1 (GLP-1). Other parameters are measured in the blood samples including insulin and glucose.

B. Effect of Preload on serum lipoproteins. The lipoprotein pattern in serum is measured in patients after 1-2 weeks of treatment with a Preload according to Examples 1-2. Around 15 patients with GDM are given preload in addition to conventional non-pharmaceutical GDM treatment. After 1-2 weeks of treatment serum is collected for lipoprotein determinations. Additional parameters are also measured including inflammatory markers, BMI and blood glucose. The measurements are continued for 1-2 months, in which every third week the BMI is measured using an impedance balance.

### Example 5. Dose Determination of Preload

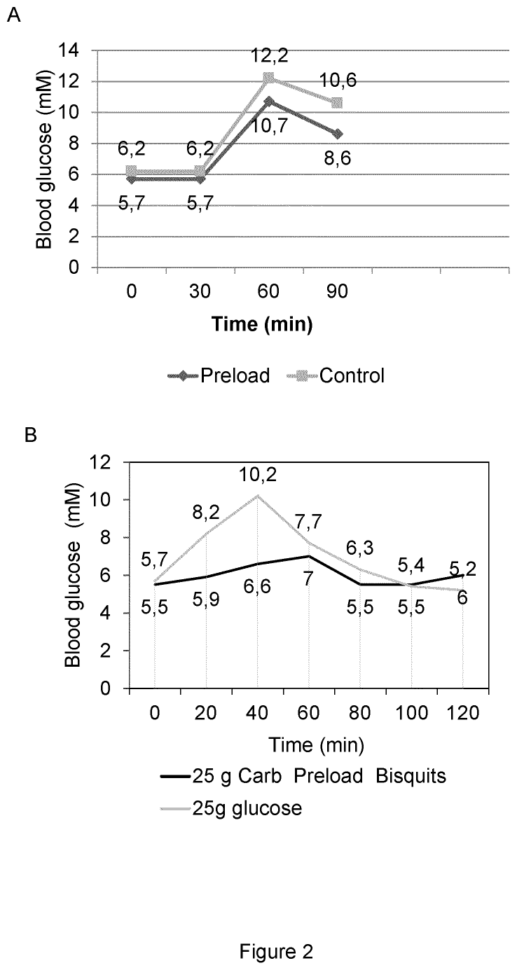

Healthy volunteers were exposed to a standard oral glucose tolerance test (OGTT) consisting of drinking a defined amount of glucose followed by repeated measurements of blood glucose during 2 h. Each individual was exposed to the OGTT a first time when no preload treatment had been administered (control) and a second time, 3 days later, after a preload treatment had been administered. A dose titration was made by testing different amounts of preload ranging from 10 g to 50 g. Each group consisted of at least three people. The principal finding was that treatment with optimized preload changed the glucose curve following OGTT notably in reducing the peak glucose value, both when a dose of 18.9 g and of 25 g was administered. The results shown in FIGS. 2A and B.

In particular in FIG. 2A: either Preload, corresponding to one biscuit total weight; 18.9 g; or Control (water) was given at time 0 on two test occasions. The Preload was made as outlined in example 1-2. The time between these treatments was three days. 30 minutes after Preload/Control treatment the subject received an oral glucose tolerance test (OGTT). Capillary blood was tested for glucose at 0 min, 30 min, 60 min and 90 min using a glucometer. It was concluded that Preload treatment reduced the glucose elevation as measured by OGTT. The experimental design is also useful for determining the dose response relationship of Preload treatment.

FIG. 2B shows the results of the test for a higher carbohydrate dose. A subject was instructed to ingest Preload in an amount that contained 25 g carbohydrates and corresponding to two biscuits. The Preload was made as outlined in Example 1-3. The effect on blood sugar was compared to the effect of ingesting 25 g pure glucose. Measurements were carried out using a glucometer on capillary blood samples taken at the time intervals shown on the X-axis. It was concluded that the intake of Preload only marginally increased blood sugar whereas the intake of glucose resulted in a robust glucose elevation.

Conclusion: Preload treatment reduced the glucose elevation as measured by OGTT.

### Example 6. Clinical Effect of the Optimized Preload on Subject with Gestational Diabetes (GDM)

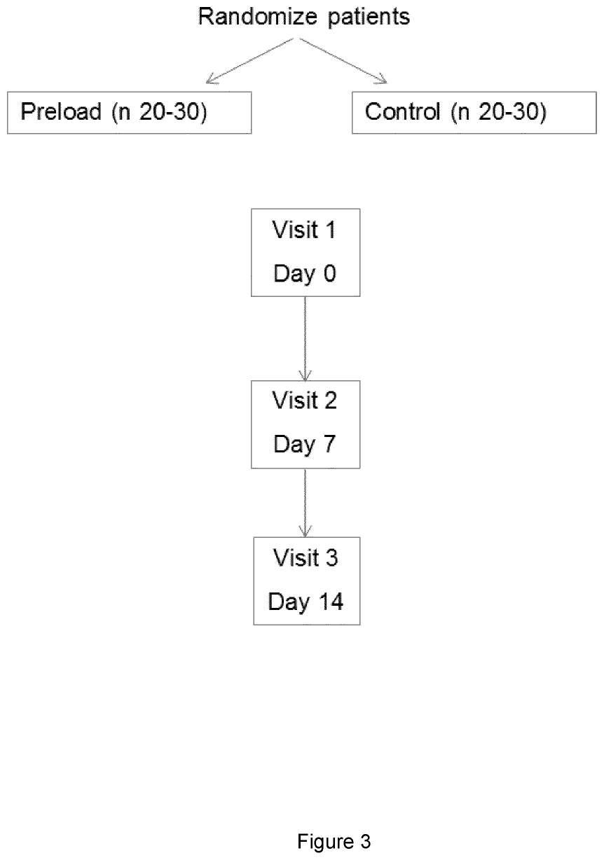

Around 50-60 GDM patients, not in need for pharmacological treatment, are recruited and instructed how to use the optimized preload. Patients affected by GDM combined with other disorders are excluded. The patients are randomized into two groups. The treatment is given in addition to standard nutritional advice. A control group consisting of the same number of patients is only given standard care. A follow up OGTT is conducted in both groups after one week. Blood sugar is subsequently continuously monitored until term. Key measurements include OGTT, change in glucose fluctuation, levels of glycated hemoglobin (HbA1c), inflammatory markers such as IL-1β, IL-6, IL-10, TNF-α, C-reactive protein (CRP), monocyte chemoattractant protein (MCP)-1, plasma level of endotoxins, Apgar score and fetal weight. An outline of the clinical study is provided as FIG. 3:
Visit 1 (Day 0): Base line assessment comprising clinical examination, body composition (impedance balance), OGTT, blood sample collection for measurements routine clinical chemistry and inflammatory markers. A continuous glucose measuring device is applied on each patient. Preload/control treatment is initiated and the same dietary advices are given to both groups.
Visit 2 (Day 7): Follow up meeting and interview. Body composition (impedance balance) and OGTT analyses. Continuous glucose readings are collected.
Visit 3 (Day 14): Final assessment comprising clinical examination, body composition (impedance balance), OGTT, blood sample collection for measurements routine clinical chemistry and inflammatory markers. Continuous glucose readings are collected. A questionnaire to assess diet, experiences, hunger and other parameters is provided to both groups.

Conclusion: The above outline is a feasibility test where the compliancy to treatment is monitored. It also documents rapid effect of preload treatment on blood sugar values and on inflammatory mediators.

### Example 7. Clinical Trial on the Effect of the Optimized Preload on Subject with Gestational Diabetes (GDM)

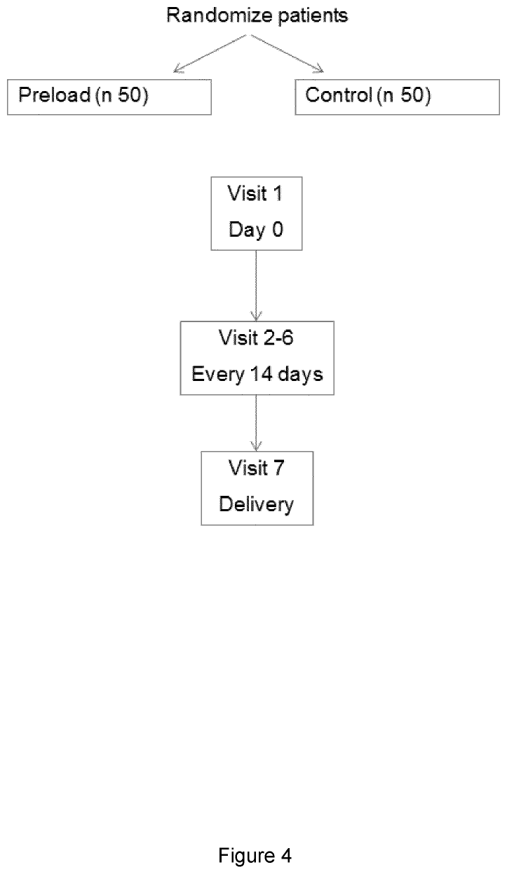

This example outlines a clinical trial to evaluate the effect of optimized preload on subject with GDM. A group of 50 GDM subjects is given preload and this group is compared to a control group of 50 GDM subjects who are not administered the Preload treatment. Patients affected by GDM combined with other disorders are excluded. The patients are randomized into two groups. The study is conducted as depicted in FIG. 4 and explained here below:
Visit 1 (Day 0): Base line assessment comprising clinical examination, body composition (impedance balance), OGTT, blood sample collection for measurements routine clinical chemistry and inflammatory markers. A continuous glucose measuring device is applied on each patient. Preload/control treatment is initiated and the same dietary advices are given to both groups.
Visit 2-6 (Every second week): Follow up meetings and interviews. Body composition (impedance balance) and OGTT analyses. Continuous glucose readings are collected. Other conventional treatment routines.
Visit 7 (Delivery): Final assessment comprising clinical examination, body composition (impedance balance), OGTT, blood sample collection for measurements routine clinical chemistry and inflammatory markers. Continuous glucose readings are collected. Birth weight and Apgar score are recorded. A questionnaire to assess diet, experiences, hunger and other parameters is provided to both groups. Other routine examinations, investigations and report.

The treatment is given from the time of diagnosis until term. The clinical management of the patients follow conventional treatment routines. In addition to regular blood sugar measurements blood samples are set aside for the analysis of inflammatory biomarkers. The delivery and the conditions of the newborns are carefully evaluated.

Conclusion: Preload treatment has positive effect on the pregnant state. The primary read out is improvement in glucose levels and secondary outcome refers to improvements during delivery e.g. reduced complications and reduced birth weight.

### Example 8. Clinical Trial on the Effect of the Optimized Preload on Overweight or Obese Subjects

This example outlines a clinical trial to study the effect of optimized preload on obese or overweight subjects. The subjects are pregnant women having increased BMI and/or overweight or obesity, but no need for pharmaceutical treatment. Patients affected by other disorders are excluded. The study design is similar to the one described in Example 7, except that
the preload/control treatment starts earlier in gestation since overweight is detected at the first visit, and
a primary read out is body composition.

The other read outs are as described for Example 7.

Conclusion: It is expected that the increase in body weight during pregnancy is less in the preload group compared to control group although both groups are given the same dietary advice. A positive outcome on delivery parameters is also expected. The main read out is body composition and secondary outcome are factors related to the delivery e.g. mode of delivery, birth weight and complications and conditions of the child.

### Example 9. Method of Preparing Preload Biscuit

Example 9 provides non-limiting examples of methods for preparing preload biscuits. Preload Biscuits are prepared using a step wise procedure as outlined below. There are three main components consisting of: 1) Processing of oat, 2) Mixing of other ingredients and 3) Baking. Consideration of these three components are important for the present invention where we have successfully been able to manufacture biscuits characterized by a reduced loss of beta glucans, reduced levels of (dietary derived) advanced glycated endpoints ((dAGE/)AGE), reduced level of phytatic acid, reduced activity of lectin and reduced activity of lipases.

The preload biscuit preferably comprises the 7 basic ingredients shown in Table 1A.

Several methods were used to evaluate and optimise the manufacture method of Preload biscuits according to the present invention. Biochemical methods included commercial assays to measure beta glucans using a beta-glucan kit from Megazyme (Bray, Ireland)(see Example 10), AGE using a AGE-Competitive ELISA kit (Cell Biolabs Inc. San Diego US)(see Example 12), phytatic acid using a Phytic acid, kit from Megazyme (Bray, Ireland)(see Example 10), lipase activity using Lipase Activity Assay Kit (Sigma Chemical, St. Louis, US)(see Example 10) and lectins using Lectin—hemagglutination test (Innnovative Research, Novi, Mich., US)(see Example 13).

Based on the findings described in Examples 10 to 13 an optimized procedure for the manufacture of Preload Biscuits is described below in this Example as “Method 2”. The product is a biscuit with acceptable taste, good levels of beta glucan, low levels of AGE, lectin and phytatic acid Another example of a useful method for manufacture of Preload biscuits is described as Method 3.

Ingredients are listed in Table 5:

TABLE 5
Baker' Percentage (according to oat 25 amount)

| | Amount (g) | |
|---|---|---|
| Oats | 100% | 40 |
| Water | 37.50% | 15 |
| Beta-Glucan | 25.93% | 10.372 |
| Salt | 1.11% | 0.444 |
| Agave syrup | 16.70% | 6.68 |
| Coconut oil | 27.78% | 11.112 |
| Vanilla | 0.74% | 0.296 |

Dependent of the size of the biscuits, the indicated amount is suitable for making about three oat biscuits.

The methods for preparing Preload biscuits used standard food processing equipment including oven, grinder and water baths.

Method 1 comprises the steps of:
1. Providing dehulled oats
2. Soak oats at room temperature
3. Germinate oats for 64 hrs
4. Heat treatment of oats in microwave for 45 s at 1000 W
5. Blend/grind oats
6. Add other ingredients
   a. Agave syrup
   b. Coconut oil
   c. Flavouring
   d. Salt
7. Mix all ingredients well
8. Add mixture to mold
9. Bake biscuits for 90 min. at 90° C. followed by baking at 135° C. for 7 min.

Method 2 comprises the steps of:
1. Providing dehulled oats
2. Submerging oats at room temperature for 6 h
3. Germinate oats for 23 h at 16° C. under 80% moisture
4. Heat treatment of oats in microwave at 1100 W for 20 sec followed by stirring and additional 25 sec and stirring
5. Weigh germinated and heat treated oats and add water till total weight is 2.3. times weight of oats
6. Grind to form a slurry
7. Add all other ingredients
   a. Agave syrup
   b. Coconut oil
   c. vanilla
   d. Salt
   e. Optionally add phytase
8. Mix until homogenous
9. Rest for 10 min.
10. Flatten to 7 mm
11. Use a 70 mm circular cookie cutter to cut out cookies
12. Bake biscuits for 1 hrs and 10 min. at 90° C. followed by baking at 135° C. for 7 min.

Method 3 comprises the steps of:
1. Providing dehulled oats
2. Submerging oats at room temperature for 6 h
3. Germinate oats for 23 h at 16° C. under 80% moisture
4. Heat treatment of oats in microwave at 1100 W for 20 sec followed by stirring and additional 25 sec and stirring
5. Weigh germinated and heat-treated oats and add water till total weight is 2.3. times weight of oats
6. Grind to form a slurry
7. Add all other ingredients
   a. Agave syrup
   b. Coconut oil
   c. vanilla
   d. Salt
8. Mix until homogenous
9. Rest for 10 min.
10. Flatten to 7 mm
11. Use a 70 mm circular cookie cutter to cut out cookies
12. Bake biscuits for 10 min. at 135° C. followed by baking at 90° C. for 1 hour and 30 min.

### Example 10

Several methods were used to evaluate and optimise the manufacture method of Preload biscuits according to the present invention. Biochemical methods included commercial assays to measure beta glucans using a beta-glucan kit from Megazyme (Bray, Ireland)(see Example 10), AGE using a AGE-Competitive ELISA kit (Cell Biolabs Inc. San Diego US)(see Example 12), phytatic acid using a Phytic acid, kit from Megazyme (Bray, Ireland)(see Example 10), lipase activity using Lipase Activity Assay Kit (Sigma Chemical, St. Louis, US)(see Example 10) and lectins using Lectin—hemagglutination test (Innnovative Research, Novi, Mich., US)(see Example 13).

Experimental tests were conducted to optimize conditions for oat processing. Dehulled oat grains were soaked in water and allowed to germinate. Different soaking times (range 2.6 h-9.4 h), different soaking temperature (range 24° C.-34° C.) and different germination times (range 0 h-64 h were tested. The germinated oat was processed into biscuits as described in Method 1 of Example 9. Samples of the oat grains and the biscuits were taken throughout the process for determination of beta-glucan and phytic acid. The beta-glucan content and the phytic acid content was determined using a beta-glucan kit from Megazyme (Bray, Ireland) and a Phytic acid, kit from Megazyme (Bray, Ireland) according to manufacturer's instructions.

Since several parameters were analyzed in several different conditions, the results were evaluated using a central composite design (Minitab 8).

The different soaking times (range 2.6 h-9.4 h), different soaking temperature (range 24° C.-34° C.) and different germination times (range 0 h-64 h) affected Beta Glucan and Phytic Acid levels in the germinated oat. One outcome of this set of experiments was that a soaking time of 6 h, a soaking temperature of 24° C. and germination time of 22 h was the most optimal to maintain high levels of Beta Glucan and to obtain low levels of Phytic Acid. A similar experiment was made, where phytase was added to the grinded oats together with the other ingredients. It was found that addition of Phytase (10% of oat amount) at the stage where other components are added lead to a complete loss of Phytic Acid in the biscuits.

The level of phytic acid in germinated oat grains prepared by soaking oat grains for different amounts of time at 24° C. with germination time of 22 h are shown in Table 6. The level of phytic acid found in biscuits relates to the level found in the germinated oat grains used for preparation of the biscuits. The level of reduction of Phytic Acid reached 85% from a defined baseline, where 100% is measured to 1.05 gr Phytic Acid/100 gr of oats when grains were soaked for 9.36 hrs. However already after 4 hrs soaking a significant reduction was obtained. As noted above, 6 hours soaking was considered optimal even though the level of phytic acid can be further reduced by longer soaking, because after longer soaking significant amounts of beta-glucan is lost.

TABLE 6

| | Phytic acid | | |
|---|---|---|---|
| | content | | |
| Soaking | (g/100 g) after | Loss of | Standard |
| Time | soaking, | phytic | deviation |
| (hrs) | mean value | Acid (%) | (g) |
| 4 | 0.37 | 64.76 | 0.26 |
| 6 | 0.43 | 59.05 | 0.18 |
| 8 | 0.42 | 60 | 0.32 |
| >9.36 | 0.15 | 85.56 | N/A |

The content of Phytic Acid measured in oats has been varying from 1,05-1.20 g/100 g of oats. Hence, calculation of reduction has been set at 1.05 g to represent 100%.

### Example 11

Experiments were made to find conditions where lipase activity was reduced. It was found that lipase activity was efficiently reduced by heat treatment (microwave for 45 seconds) of oat at the germination stage (FIG. 6).

Raw dehulled oats were soaked for 6 to 8 hours at 24° C., and germinated for 64 hrs. The germinated oats were heat treated by one of the following methods:

Drying in combi-oven at 100° C. for 90 min.

Microwaved at 1100 W for 45 seconds.

The lipase activity was measured in samples corresponding to 50 g raw oats using the lipase activity assay kit from Sigma Chemical, St. Louis, US according to manufacturer's instructions.

The result is shown in FIG. 6.

Heat treatment reduces lipase activity significantly regardless of whether treatment is performed by drying in the oven or by microwave treatment.

Thus microwave treatment, which is a fast procedure, reduces lipase activity. Preferably, heat treatment should be carried out immediately after germination to prevent lipid oxidation and rancidity. Strength and duration is dependent on amount of oats.

### Example 12

Formation of Advanced Glycation End Products (AGE) during baking procedures is unwanted in Preload biscuits. Experiments were conducted to test the effect of different baking conditions on levels of AGE. As can be seen in Table 7 a low baking temperature (70° C.) for 30 min lead to a significantly reduced level of AGE in Preload Biscuits.

Biscuits were prepared essentially as described in Example 9, Method 2 except that different baking conditions were tested.

Different baking conditions with baking temperatures in the range of 70° C.-110° C. and baking times in the range of 30 min to 2.5 hours were tested as specified in Table 6.

AGE was analysed using an AGE-Competitive ELISA kit (Cell Biolabs Inc. San Diego US) according to manufacturer's instructions. The baked products were mixed in dilution buffer (50 mM Tris-HCl ph 7.4 and 0.05% tween 20) for 5 mins in a vortex mixer before detecting AGE.

The results are shown in Table 7. Results are expressed as kU.

TABLE 7

| | Conditions | Overall (AGEs,kU) |
|---|---|---|
| trial 1 | 90° C., 90 min | 20.18 ± 1.11 |
| trial 2 | 70° C., 30 min | 13.99 ± 3.81 |
| trial 3 | 110° C., 30 min | 20.34 ± 3.62 |

Reducing time and temperature during baking leads to a reduced formation of AGE. The lowest level of AGE in this experiment was when a baking temperature of 70° C. for 30 minutes was used.

### Example 13

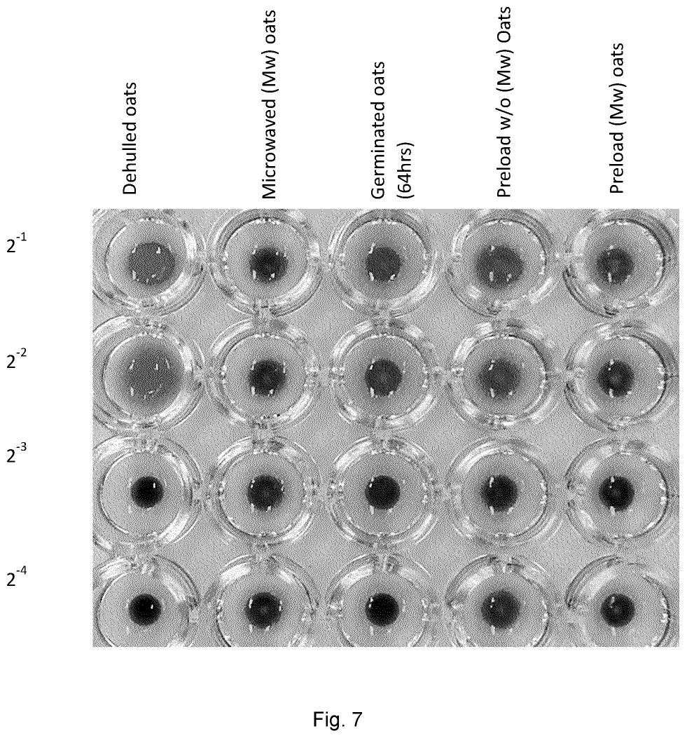

The presence of lectin molecules is another unwanted component in Preload biscuits. As shown in FIG. 7, microwave treatment reduces lectins in Preload biscuits.

Preload biscuits were prepared as described in Example 9, method 2 (Preload with microwaved oats). In addition. samples were regularly taken during the method. Thus, a sample of raw oat prior to any treatment (Raw oat), a sample of the oat just after germination (Germinated oat) as well as a sample after microwave treatment of the oat (Microwaved oat) were also analysed. In addition one preload biscuit was prepared from oat, which had been malted according to method 2 of Example 9, but which had not been subjected to heat treatment (Preload without microwaved oats). The lectin content was tested for using the Lectin—hemagglutination test (Innnovative Research, Novi, Mich., US) according to manufacturer's instructions. The method is semi-quantifiable, but does measure specific end-levels.

The results are shown in FIG. 7.

Lectin is Present in
raw oat (Column 1),
germinated oat (Column 3)
Preload without microwaved oats (column 4)

No Hemagglutination
Microwaved oat (column 2)

Vague Signs of Hemagglutination
preload with microwaved oats (column 5) showed signs of the presence of lectin despite the oats having been microwaved.

The preload biscuit include other ingredients such as oat fiber, that was added after the heat treatment. It is possible that lectin could have come from the fibre which is made from whole grain oats (Promoat™; Biovelop, Kimstad, Sweden).

### CONCLUSION

Heat treatment results in significant reduction of lectin in the preprocessing stage. In fact no lectins were detected in microwaved oats.
However, it is to note that other raw ingredients may contain a small amount of lectin

### Example 14

Clinical trial to test preventive effects of Preload Biscuits on GDM Gestational diabetes (GDM) is defined as diabetes discovered during pregnancy. It is most common to diagnose GDM at mid-term or the last trimester. The aim of the study is to investigate if Preload biscuit can prevent GDM. The Preload biscuit may be prepared according to Method 2 or Method 3 of Example 9, and each biscuit may be 27 g. GDM is diagnosed in 5-25% pregnant women By using selection criteria for high risk assessment consisting of ethnicity, age, BMI and previous GDM pregnancy, patients with a high risk to develop GDM are selected and the expected incidence in this selected group is predicted to be in excess of 70%.

Clinical Trial Outline

High risk patients (risk assessment, high BMI, age etc) determined at the beginning of pregnancy (first visit to maternity care) are randomized into a control group and a treatment group and checked with Hb1AC.

The subjects are treated with Preload (see details below) until blood glucose measurement is performed during a routine follow up appointment in the late second or early third trimester. The blood glucose measurement may be Oral Glucose Tolerance Test (OGTT) or a similar test.

Primary readout I is blood glucose measurement (e.g. by OGTT) after treatment compared to controls

Primary readout II is Hb1AC before and after treatment compared to controls

Secondary readout is BMI before and after treatment compared to controls

Secondary readouts further concern influence on fetal development, especially fetal weight since GDM may give rise to fetus large for gestational age (LGA), and macrosomia.

Procedures.

Pregnant subjects (n=160; min 120) with an increased GDM risk are selected using the described inclusion criteria. Subjects are asked if they want to take part in a clinical Preload investigation. This will mean to ingest one Preload biscuit of approx. 27 g prepared as described in Example 9, Method 2 or 3 three times/day one half-hour before each meal until a blood glucose measurement is carried out. The study randomizes subjects into two groups to create a two armed randomized, not blinded clinical study. Controls will not be treated with placebo. The plan is to incorporate the study in routine procedures at maternity special care units.

Inclusion criteria
Normal oral glucose tolerance test (WHO criteria)
Age 25
High risk assessment as in procedure

Exclusion criteria
Underlying chronic disease
Need for insulin/anti-diabetic medication
Other medications, drug-abuse, cognitive disturbances

Schedule outline

| Activity | Pregnancy week | Treatment duration |
|---|---|---|
| First visit to maternity care unit | | 0 |
| Routine baseline HB1Ac, BMI | | 0 |
| Selection of study participants and | | 0 |
| information | | |
| Start of Preload treatment | | 0 |
| Second follow up blood glucose | 25-28 | 22-26 |
| measurement, HB1Ac, BMI | | |

Sample Size and Power.

A power calculation (0.8) was carried out using a significance level of 0.025, SD of 1.29 and a difference in means of 1. This resulted in that minimally 56 patients should enter this two armed (active and control). This figure has to be increased to make room for drop outs or for patients in need of insulin/drug treatment and for subject not developing GDM. The dropout rate is estimated to be low because of highly motivated patients. An estimate is that 120 subjects are tested, 60 in each group.

### Example 15

Preload Biscuits Stabilizes Blood Glucose Levels

The effect of Preload biscuits was tested in 3 healthy volunteers as outlined below.

Test 1

The volunteers fasted for 8 hours after which they ate 2 Preload biscuits of 18 g each together with 200 ml water. The blood glucose level was determined at regular intervals as outlined in Table 8 below.

Control 1

As a control the volunteers fasted for 8 hours after drank 200 ml water. The blood glucose level was determined at regular intervals as outlined in Table 8 below.

Test 2

The volunteers fasted for 8 hours after which they ate 2 Preload biscuits of 18 g each together with 200 ml water. After another 30 min they ate 25 g glucose. The blood glucose level was determined at regular intervals as outlined in Table 8 below (0 min. being the time of glucose intake).

Control 2

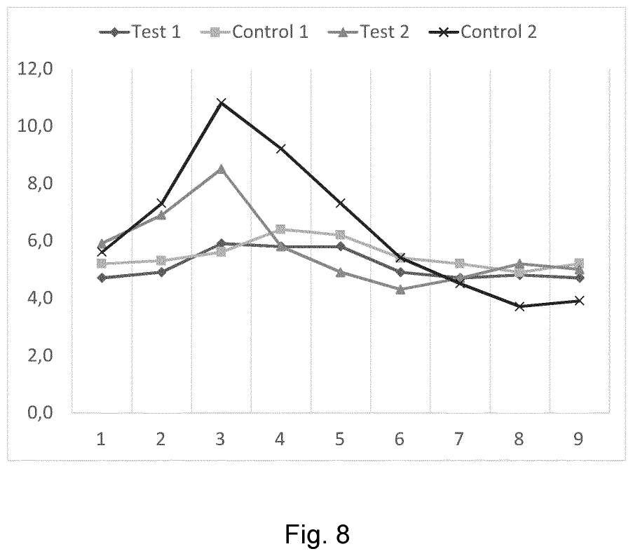

As a control the volunteers fasted for 8 hours after which they drank 200 ml of water. After another 30 min they ate 25 g glucose. The blood glucose level was determined at regular intervals as outlined in Table 8 below (0 min. being the time of glucose intake). The results are shown in Table 7 below as well as in FIG. 8.

TABLE 7

| | Test 1 | | Control 1 | | Test 2 | | Control 2 | |
|---|---|---|---|---|---|---|---|---|
| | Blood | | Blood | | Blood | | Blood | |
| Min- | glucose | Std | glucose | Std | glucose | Std | glucose | Std |
| utes* | mmol | dev | mmol | dev | mmol | dev | mmol | dev |
| 0 | 4.7 | 0.89 | 5.2 | 0.55 | 5.9 | 0.63 | 5.6 | 0.55 |
| 15 | 4.9 | 1.00 | 5.3 | 0.77 | 6.9 | 1.00 | 7.3 | 0.55 |
| 30 | 5.9 | 0.84 | 5.6 | 1.05 | 8.5 | 1.38 | 10.8 | 1.48 |
| 45 | 5.8 | 0.90 | 6.4 | 0.63 | 5.8 | 1.09 | 9.2 | 1.30 |
| 60 | 5.8 | 1.09 | 6.2 | 0.89 | 4.9 | 0.77 | 7.3 | 1.07 |
| 75 | 4.9 | 0.77 | 5.4 | 0.74 | 4.3 | 1.09 | 5.4 | 0.89 |
| 90 | 4.7 | 0.80 | 5.2 | 0.54 | 4.7 | 0.89 | 4.5 | 0.71 |
| 105 | 4.8 | 0.87 | 4.9 | 0.81 | 5.2 | 0.54 | 3.7 | 0.22 |
| 120 | 4.7 | 1.14 | 5.2 | 0.89 | 5.0 | 1.00 | 3.9 | 0.22 |

*Approx. number of minutes

The results show that intake of Preload biscuits alone did not have a significant impact on blood glucose levels, however, intake of Preload biscuits 30 min prior to intake of glucose resulted in a much lower rise in blood glucose levels, and furthermore it also resulted in a lower decrease in blood glucose levels. Thus, intake of Preload biscuits resulted in smaller fluctuations in the blood glucose levels.

### REFERENCES

Kletter D, Curnutte B, Maupin K A, Bern M, Haab B B (2015). Exploring the specificities of glycan-binding proteins using glycan array data and the GlycoSearch software. Methods Mol Biol. 1273:203-14.
Makower R U (1970). Extraction and determination of phytic acid in beans (Phaseolus vulgaris). Cereal chemistry 47:288-295.
Miquel-Kergoat S, Azais-Braesco V, Burton-Freeman B and Hetherington M M (2015). Effects of chewing on appetite, food intake and gut hormones: A systematic review and meta-analysis. Physiology & Behavior 151:88-96.
Jian V., Pitchumoni C. S. J. (2009) Gatrointestinal Side Effects of Prescription medications in the Older Adult, Gastroenterol 43 (2), pp 103-110 Wheeler E L and Ferrel R E (1971). A method for phytic acid determination in wheat and wheat fractions 48:312-320.

1. A method for manufacturing a solid food composition, the method comprising:
A. Providing cereal grains;
B. Treating said grains to reduce their phytic acid content;
C. Subjecting said grains to a step of heat treatment;
D. Finely dividing said grains
E. Adding at least one liquid and one or more additional ingredients to said finely divided grains to obtain a slurry
F. Incubating the slurry at a low temperature in the range of between 60° C. and 95° C. for a time interval in the range of between 30 and 180 minutes; and
G. Incubating the slurry at a high temperature in the range of between 125° C. to 140° C. for a time interval in the range of between 1 and 10 minutes;
wherein step B. may be performed at any time during the method and steps F. and G. may be performed in any order, thereby obtaining a solid food composition.

2. The method according to claim 1, wherein the cereal grains are dehulled oat grains.

3. The method according to any one of the preceding claims, wherein step B. comprises the steps of
B1. submerging said grains in water
B2. germinating said grains.

4. The method according to any one of the preceding claims, wherein step B. is performed in a manner reducing the level of phytic acid in said grains to less than 70%, preferably to less than 60%, such as to less than 50% of the initial level.

5. The method according to any one of the preceding claims, wherein the additional ingredients are one or more selected from the group consisting of salt, sweeteners, vegetable oil and soluble fibres.

6. A solid food composition manufactured by the method according to any one of the preceding claims.

7. A solid food composition comprising:
a protein in a range between 3 and 18% dry weight,
a lipid in a range between 8 and 25% dry weight,
a complex carbohydrate in a range between 35 and 80% dry weight,
soluble fibers in a range between 2 and 19% dry weight
wherein the complex carbohydrate has been treated to comprise a reduced amount of phytic acid compared to the untreated carbohydrate, and
wherein all ingredients of the composition are of plant origin.

8. The composition according to any one of the preceding items, wherein the complex carbohydrate is comprised in grains of a cereal or a pseudocereal, and the composition comprises said grains or parts thereof.

9. The composition according to any one of the preceding items, wherein said composition has a level of phytic acid below 5‰, preferably below 4‰, such as below 3‰.

10. A solid food composition according to any one of the preceding items for use in the treatment or prevention of a metabolic disorder.

11. The solid food composition according to claim 10, wherein the metabolic disorder is selected from the group consisting of obesity, diabetes type II, gestational diabetes mellitus, polycystic ovary syndrome (PCOS), androgen deficiency in a male individual and any combinations thereof.

12. The solid food composition according to any one of the preceding items for use in a method of reducing blood sugar levels, blood sugar fluctuations, low-density lipoprotein (LDL) cholesterol, insulin fluctuations, and/or BMI in an individual.

13. The solid food composition according to any one of the preceding items for use in a method of reducing inflammation in an individual.

14. The solid food composition according to any one of the preceding items for use in a method of normalizing a dysbiotic microbiota in an individual.

15. The solid food composition according to any one of claims 7 to 10, wherein said composition further comprises a drug.

16. A kit-of-parts comprising the solid food composition according to any one of claims 7 to 10 and a drug.

17. The composition or the kit-of-parts according to any one of claims 15 to 16, wherein said composition or said kit-of-part is for use in a method of reducing gastro-intestinal side effects of said drug.
